SEQUENCE LISTING (82 sequences)

SEQ ID NO: 1 - DNA, 1245 bp, Abelmoschus esculentus
  source 1..1245: mol_type = genomic DNA
atgtgcttactcaacaaggtggcgcagcagagaggttcaagtgactacgccaggtttcctactgatggagatggtggcggtgccgaagatgaacaacaatattcccagttgtctcaaatccaacaactctaccgtccgatgatgcaacatcggaacacagctgaaatgttttttggacaacagggtcaatcatcaaacgagatgtctgcaatggtacctgttggatcgggacaaagggcagctgctgattggggttatgttgctaacatagcaggcaaggtctcatctggttttgcacacagcagctcaccagctcgttcttcaccttctggttctggttcgagttctggtatatgggttggtaaaaaacgagggcgtgaagatgaggctagtgttcatttcattgagtcttcaccgagaggggttcaaagaacttttgtcgattttagaggcgagtcagcatcagctgcagctgccgttgctgaagaaacaacgagcattgttgcatcggcagcagaaacaacagtaggagcagcggccgtggcatcctatgaagaaagcggcgagcgaagaagaagatatagaggcgtaaggcaaaggccatgggggaaatgggctgcagaaatacgcgatcctcataaagcagcaagagtctggctcggcacctttgacaccgcagaagctgccgcaagagcctacgatgaagccgccttgagattcaggggaaacagggccaaattaaatttccccgagcttgtcaggtctgttcctcaaccaatgcagaacttccccgctacccaaacaccagtttctacttctcttccaacccatttccaatcttctaatccaacaccattgccgtcgttttaccaaccccaaactcttcagagctccactgctgatatgctgagggattactggcagtactctcagttgctgcagagttcttccggtgtccatggacaacaagatacgaggttgatggaacagatgatccaatcctctcaaatacctaatattcaacagccattgttatcttcttctttatcacctttcccttcttcatttgtagcttcctccggttcttcatcttcttcctctgcttattttcctgtgcttttctccgagcatcaacaaatggacattttcgggcagccgtccaatcaaactcaggcaagtgggtcagattttccagtgccaccatggtctcatcccgatcatgatccttcatccacaggttcataa

SEQ ID NO: 2 - protein, 414 aa, Abelmoschus esculentus
MCLLNKVAQQRGSSDYARFPTDGDGGGAEDEQQYSQLSQIQQLYRPMMQHRNTAEMFFGQQGQSSNEMSAMVPVGSGQRAAADWGYVANIAGKVSSGFAHSSSPARSSPSGSGSSSGIWVGKKRGREDEASVHFIESSPRGVQRTFVDFRGESASAAAAVAEETTSIVASAAETTVGAAAVASYEESGERRRRYRGVRQRPWGKWAAEIRDPHKAARVWLGTFDTAEAAARAYDEAALRFRGNRAKLNFPELVRSVPQPMQNFPATQTPVSTSLPTHFQSSNPTPLPSFYQPQTLQSSTADMLRDYWQYSQLLQSSSGVHGQQDTRLMEQMIQSSQIPNIQQPLLSSSLSPFPSSFVASSGSSSSSSAYFPVLFSEHQQMDIFGQPSNQTQASGSDFPVPPWSHPDHDPSSTGS

SEQ ID NO: 3 - DNA, 936 bp, Abelmoschus esculentus
  source 1..936: mol_type = genomic DNA
atgtccgctgctttcacaacttcggctacaaatgttttcgggttcggaagctacaccaccgtgcttcaaaaggtggattcggtgtcgacttcttcagtgcccgtcccaccaccaaagccactccttattgccacacctagtgaagcaggagaattcccactactcatattcctccatggttaccttctttacaactctttctactctcagcttctccagcatgttgcttcccatggtttcattgttattgctcctcagttatacaccgtggccggaccggatgcgaccgatgagattaagtccacagctgcaataaccatttggttatccaaaggactactccaagggttgcttccacaagatgttcagccaaacttaagcaagctcgcacttgccggtcacagcagaggaggcaaagttgctttcgcgctagctctgcagaaagccatgaccacgttgaagttttcggccttaataggtgtagaccccgtcgatggaatggataaagggaaacaaacgccgcctccggtactcacttatgttcctcattcgttcgatcttgatatggcggtaatggttatcggttcgggtctcggtgatgttaggagaaatccattgttccctccttgtgcacccaagggagttaaccatgaagatttcttcaaagaatgtagaaaaccggcatgtcatttggttgccaaggactatggtcatctcgatatgttggatgatgagactaaaggtttaagaggacgttcatcgtattgtttgtgcaaaaatggcaaggctcgagagccgatgagaaggtttgtcggaggagccattgttgctttcatgaaagcctatcttaatggagacaagactgaattgattgctataaaacatgggcatgaaactgcaccggtggagcttgaaacgacggaatttcttgtttaa

SEQ ID NO: 4 - DNA, 936 bp, Abelmoschus esculentus
  source 1..936: mol_type = genomic DNA
atgtctggtgctgtcacagattcgggcaccaatgtttttgggtttggaagctacaccactgtgcttcaaaaggttgactcagtacccactcgttcagtggctgacccacccccaaaaccactcctaattgcaacaccttctgaagctggagaattcccgctactcatcttcctccatggctatcttctgtacaattctttctactctcagcttctccaccatgttgcttcccatggcttcattcttattgctcctcagctttacaccgtggctggaccagatgcaaccgatgaggttaagtccacggctgcaataaccaattggttgtccaaaggagtactccaagggttgcttccatcggatgttcgcccgaacttaagcaagcttgcactttccggacacagtagaggaggcaaagtcgctttcgcactagctctgcagaaagcaatgactacgttgaagttttcggccttaataggcgtagaccccgtcgatggaatggataaagggaaacaaacgccgcctccggtacttacctatgttccgcattcgttcgatcttgatatggcggtgatggttatcggttcgggtcttggtgaagttagaaagaatcctttgtttcctccttgtgctcccaagggagttaaccatgaagatttcttcaaagaatgtaggaaaccggcctgtcatttcgttgccaaggactatggtcatctcgatatgttggacgatgagactaaaggattacgaggacgttcgtcatattgtttgtgcaaaaatgggaaggctagggagccaatgagaaggtttgttggaggagttcttgttgctttcatgaaagcttatcttaatggagataacaccgacttgattgctataaaacatgggcatgaaacggcaccggtggagcttaaaacggtcgatttccttgtttaa

SEQ ID NO: 5 - DNA, 1101 bp, Abelmoschus esculentus
  source 1..1101: mol_type = genomic DNA
atggccactgcttcaacttgccaattcttcactgatttttggtcttcaagggctcatccaatcatcttctcacccatctccaaaccctcccattttcagtcttacagccttacagctaatgtttcacttcaaaagaaccacagcctctccaatcctccactgctagtaaaagctcaagcttccatcaatggaaaaacgcaacccatggttcctccttacaacgttctcataactggctcaagcaaaggaatagggtacgccttggcgaaaaactttctggtagctgatgacaatgtcatcatttgttcaagatctgctgaaggggtggaatccgcttttaagagcctcagagaagaatatggggaacgtgtgtggggtactcagtgtgatgttagagtggcagaggatgtgaagaatttagttttattcgctcaaaagaacctgggatacattgacatatggattaataatgcaggatccaatgcatatagttataaaccactggcagaagcatcagatgaagatcttattgaagttgtttctacaaacacccttgggttgatgatttgttgtcgtgaggccatcaagatgatgttgcaacagcctcgaggtggtcatattttcaacattgatggagctggttcagatggaagaccaacccctaggtttgctgcatatggtgcaaccaaacgtagcgtggttcatttgacaaaatcgctgcaggctgaactgaagatgcaagatgttaaaaatgttgtaatacataatctatcgccaggaatggtgacaaccgatcttctaatgtctggtgcaaccacaaagcaggtctactatgcttcatatgcttcttttgttagttcagtttataaaagaaattttctaatattttgtcattttcaggccaagtttttcatcaatgttttagcagaaccagctgaagtggttgcagactaccttgtgccgaacatcaggtcgatcccagcaagtggatcgtggaagccaacttatatccggttcctcactggcttgaaagcctactcccagatattttcaagactagcatttggtgcaaggcgaaaccgatacatgcttgaagattga

SEQ ID NO: 6 - DNA, 723 bp, Abelmoschus esculentus
  source 1..723: mol_type = genomic DNA
atggtaactttaacttttgctcctgttttgcctgctaacacaagactgtcggtgtttgaactcaatggacccctcttcgtttccagaagaaaacccaagaagatgaaccaagctgttgtccctgtggcaaggttgtttgggccagctatattcgaggcatcgaagctaaaggtattgttcttgggagttgatgaaaagaagcacccagggaagctaccaagggcttacacccttacgcatagtgatataacagctaaactcactttagccatctctcaaaccataaacaactctcagttgcagggatgggcaaacagaatatacagagacgaagttgtcgcggaatggaagaaagtgaagggaaagatgtctttgcacgtccactgtcacataagcggcggccatttcctgctagacttgtgtgctagacttcgttacttcatcttctgcaaagaacttcccgtggtactgaaggcgtttgtccatggagatgggaacctgctgaaaaactaccagaactgcaggagtcgttggtttgggtttattttcactcgaacgtgccggagttcaacagggtggaatgctggggtccgctggtggaagcgcagcacctctagcggcggcgatggcggttgtgatgggaaaagacaagaggtgttggaagcgagtgggaatggcctcagcggtgcggagatgagtgtaattgttgctttccgccaatga

SEQ ID NO: 7 - DNA, 1569 bp, Abelmoschus esculentus
  source 1..1569: mol_type = genomic DNA
atggctgcactggcaaaacttcacttacgtagtttccagaaagaccaaccgttttctttcaaggaacaaccgcggagctggattaaacggtgccgttttctacggttgcccatcataacgagaaacacttatggggtgtacgttcaaaggtgtcggtcattcaggggagactatggagggggagcagtgggagagaaagagatggaaagtgcgggaaaagttgggattttgatggaaaatgaagtgaagttggaaacaggagatgggttttggaaatctttgaaatctgctgttttgggggttagtaaattcgattcccaatctcaagatgaatatgaaaaaactgtagctaaagttgaaggagttttctctttgatatctatgcaaataggaagatatgtagtgaccatgatgagcactggagtgatactcttgactggttttcagctgtcaggtggagacagtgagatgaatgcattgatttggtatagctggcttggagggattataattggaaccatgattggtgctaatatggttctagaggaacactgtcgagccggtccgagaaatgttgttataacaggaagcacaaggggattgggaaaagcacttgctcgagagtttcttctttcgggagattgcgtggttgttacttctcgcagtcccgagtctgttcatgtgactgtcaaagaacttgaggaaaacctcaaggaaggtatgactgcaggtggttcatttagtaagaatctgggacgagcaaaagtggtcggcatagcatgtgatgtttgtgatcctagtgatgttgaaaaattagccaacttcgctgtgaatgaactcgggtctgttaacatttgggtaaataatgctggcactaacaaaggttttagaccgctgctacagttcagcgatgatgatattcgacagatcgtctcaacaaatcttatcggatctattctctgcactcgggaagctatgcgcataatgaataaccagccaaaggtgggacacatttttaacatggacggagcaggttccgggggatctagcactcctttgactgctgtatacggttcgactaagtgtggtcttaggcagcttcatgtatcacttctgaaggagtgcagacgttctaaagtcggaatccatactgcatctcccggcatggtcctaactgatctgctcttaagtggttcgaccttaaagaacaagcaaatgtttaatgtcatctgtgagcttcctgaaactgttgctagaagtctcgttccacgattgcgggttgtaaaggggactgggaaggtcatcaactacttgaccccacctcgaatattactagctttagtcaccgcctggctccgacaaggtcgctggtttgatgaacagggtagggccttatacgcagcagaggcggaccgcattcgaaactgggctgaaaaccgtacacgcctctcgttcacagatgcaatggatatgtacacagaaaacacctgggtgtctgtgttctcactgtcggttgtttgtgccttcattatcctttctagcacgaacagcgcctttcccggcacctga

SEQ ID NO: 8 - DNA, 1545 bp, Abelmoschus esculentus
  source 1..1545: mol_type = genomic DNA
atgacttcagctgtttcctgcttcttaagacccatcttatcttcttcttcaaaagaatcaaaccccgatgtgaagaaaacagtgaagctgagagatgactggaggaaacgctctaagcctataccccctggagggacatatccagccaaagaccattgcagtcgatgtgggttgtgtgatacatattatatttcccatgttaaggatgcttgcgctttcttaggagatggaatgtccaaaattgaaagcttagaacctgtagttcacggtagagggaggaaatcagattctttggatgagacatatttaggagtctatgaaaagctgctatatgctcgtaaagttaagcctgtagaaggagctcagtggactgggatcgttacaatcattgcaatagaaatgctgaaatcaggcatggttgaggctgttatttgtgttcagagtgatccagatgacagactgtccccaaggcccattttagctaggacaccagatgaagttttagctgctaaaggagtcaaaccaacattgtcccctaatctgaataccctcgcactggttgaggcagcaggggtaaagcgtctccttttctgtggtgttggttgccaagtgcaggcgttaagatctgtggagcaccatctaaatttggaaaagctttatgtgttgggcaccaattgtgttgataatggaactcgagaaggtctagataagtttctaaaggctgcgagtagtgaacctgaaactgttctgcattacgagtttatgcaagattacaaggtccacttaaagcatttggatggtcatattgaagaggttccttatttctgtctaccagcaaacgacttagtagatgttattgcaccttcatgctatagttgtttcgattacacaaatggattagcggatctggtggttggatatatgggcgtaccaaaatattctggaatcagcatgacgcagcatccgcaatatgttaccgttagaaacgaacgtggcagagaaatgctgagtttggttgagaaccttttggagatcactccaacaattagtagtggtaatcggaaaccatttgtgatggagactgttaaagcagatgacgatgctaagttcggtatgtatggcatcttctgtttagccatggaaacaatttttgtttcaaccgcagtgcataaacattaagcttaaatgcattatatggagtccttgtacaagtgatgataactgcatatagctgaaacttacttagaggattcatggagtatgtactacattatactgtgtattttgccttcaggaaagggtccttctcaacctgccccaaggttgattgggaatttgatagcttttgtgctcaacttgattggtccaaagggtctggaatttgctcggtattcacttgattaccatactataagaaactacttgtatgtgaaccgtgcctgggggaaacaaagagctgataggcatattccttcatacgcaaagaaacttgtggatgcttataaccagaagggtgatattgaccaaatgcttacccgtcagtaa

SEQ ID NO: 9 - DNA, 948 bp, Abelmoschus esculentus
  source 1..948: mol_type = genomic DNA
atggctgtcgtcttcccacgttttctaaattcccctctccctcactcttcacttgcttcttctttggctccgccttcacccaggcctcaattctttgcatcttctgcaccatcatcgtcatctcccatggaaatcgccccccaaaagtacacggatttcacattcgtctctgccccacataggaaccttatggttgatatggtgtcgacgatggagaatcgtctggactctcagctcaaaccatgcactctaccctctgatatacaatattactacactccaagtgggaactcccatggctcactttatatcagatcaggccatacctcttctccggttgatttcgttctaggaagttggataaactgtaagatgccaacagagggagcactgaacataacaagcctttcagcatatctgaatgcttccacagatgccccgaatttcctgattgaactcatacagagtagccctacatcacttgttttccttctggatctgccccctcgaaaggacctggtcttatatcccgagtaccttaatactttttacgagaacacccgacttgaatcacttaggcaaacgtttgagaaactcccagaagttcaaccttactactcctctgctctttacattcgatgccttacatccccaacttcaatcatgatccaaatcaatacagagggatccagagaaggacgcatggaggagattataaaggatcatgtacacccggtagcaaaagaagcattggggatatggttggaccgatgcgcttgtggacatagaaatgtggacgaggaaggaaaggcatatttggagaagagggatgggttgattaagaacaagacaattgagattgatataggttcaagcttcccaagattgtttgggcccgacatagcaaatcgtgtattagaggtgataagggaagcatacaatggctga

SEQ ID NO: 10 - DNA, 1614 bp, Abelmoschus esculentus
  source 1..1614: mol_type = genomic DNA
atggcagcctctcttcttgctaccatccctacagccaatttggctactccttttataacatcttcggaaagcaaaacccatctctttttatgtccacggaagaaccttgcttcaagaagattcacgaagcttacacctttacacgtggcagcctctccatcatcatcatcagcagcagaacgagcttcagatcagaaacaagatgaattcagggtaggggatgaatttggtgaggagagttcaaagtctaagttctcatggaaagatcattggtacccggtttctttgattgaagatttggacccatctttacccacaccatttcagcttctgggtcgtgacttggtgctttggtttgataaaggaacgagccaatgggttgcttttgatgataaatgcccccataggcttgcccctttgtccgaagggaggatagatgagaatggggacttgcagtgttcatatcatggatggtcttttgatggatgtggatcttgcactaggattcctcaggctgcgtctcaaggtcccgaagctcgtgcgaatcggtctccaagagcatgtgctactaggtttcctacaatggtttctcaaggcttgctttttgtttggcctgatgagaatgggcaggaaagagcaagtgctaccaatccccccatgttgcctgatgactttagcagacccgaatttgcgactgtgaatatccaacgtgatctgttttatggttatgataccctcatggagaacgtctccgatccttcccacatcgattttgctcatcacaaggttactgggaggagagacagagcaaagcctttgccgtttaaggtggaatctagcggcccctggggatttggtggatcaaatgaggataacccaaagatcagtagtaagtttgttgcaccgtgctattatatgaataagatagagatagacacgaagcttccaatagttggtgaacagaaatggaagatatggatatgttcattcaatgtaccaatggctccagggaagacacgatcaatcgtttgcagtgcccgaaacttcttccagttttctgtgcctgggcctgcctggtggcagctggttccgagatggtttgaacactggacatcaaacgaagtttatgatggagacatgattgtactccaaggtcaagaaaagatatttctttcaaaatcaatggagggttccaaggacatcaatcaggagtattcgaagctcacatttacaccgacacaagctgatcgattcgtcttggaattccggaaatggctaaggcgtcacggaaacagtcaacccgaatggctcaacaccaccatccaacctctcccgtcaacagtattatcaaaacgccagatgcttgatagatttgagcagcacacactcaagtgttcatcatgcaaacaagcttacacgtcgttccaaacatggaaaaagatcttagtaggtgcaactgttgcattctccgcatcagccggcgtcccatcagatttgcagtttcgggttgttttagcggtgcttgcagtgatttctgcaggtttggcatttgcattacatcaactcgaaaagaacttcgtttttgtggattacgtacatgctaaaatagattag

SEQ ID NO: 11 - DNA, 1611 bp, Abelmoschus esculentus
  source 1..1611: mol_type = genomic DNA
atggcagggtttcttcttgctaccattcctacggtcaatttggctactcctattataacatcttcggaaagcaaaacccatatttttttatgtccacggaagagccttgcttcaaaaagattcacgaagcttacacctttacacgtggcagcctctccatcatcagcagctgcagaacaaacttcagatcagaaacaagaagaatttagggtaagagatgaatttggtgaggagagttcagagtctaagttctcatggaaagatcattggtacccggtttctttggttgaagatttggacccatctttacccacgccatttcagcttttgggtcgtgacttggtgctttggtttgacaaaggaaggaaccaatgggttgcttttgatgataaatgtccccataggcttgctcctttgtccgaagggcggatagatgagaatgggcacttgcagtgttcatatcatggatggtcttttgatggatgtggatcttgcactaggattcctcaggctgcgcctcaaggtcccgaagctcgtgcaaatcggtccccgagagcatgtgctactaggtttcctacaatggtttctcaaggcttgctttttgtttggcccgatgagaatgggcaggaaagagcaagtgctaccaagccccccatgttgcctgatgactttagcaaaccagaatttgcaactgtcaatatccaacgtgatctgttttatggttatgataccctcatggagaacgtctcggatccttcccacattgattttgctcatcataaggttactgggaggagagacagggcaaagcctttgccgtttaaggtggaatctagtggcccttggggatttggtggatcaaatgacggtaacccaaagatcagtagtaagtttgttgcaccgtgctattatacgaatacgatagagatagacaccaagcttccaatagttggtgagcagaaatggaagatatggatatgttcattcaatgtgccaatggctccagggaagacacggtcaattgtttgcagtgcccgaaacttcttccagttttccgtgcctgggcctgcctggtggcagctggttccgagatggtttgaacactggacatcaaacaaagtttatgatggagacatgattgtactccaaggtcaagaaaagatctttctttcaaaatcaatggagagttccaaggacatcaatcaggagtattcgaagttcacatttacacccacacaagctgatcgcctcgtcttggaatttcggaaatggctaaggcatcacggcaacaatcaacctgaatggctcaataccaccatccaacctctcccgtcaacagtattatcaaaacgccagatgcttgatagattcgagcagcacacactcaagtgttcatcatgtaaacaagcttacacgtcattccaaacatggaaaaagatcttagtaggtgcaactgttgcattctccgcatcagccggcatcccatcagatttccagctccgggttgttttagcagtgcttgcaatggtttctgcaggtttggcatttgcattacatcaactcgaaaagaacttcgtttttgtggattacgtacatgctgaaatagattag

SEQ ID NO: 12 - DNA, 20 bp, synthetic construct
  source 1..20: mol_type = other DNA
ccgagcttgtcaggtctgtt

SEQ ID NO: 13 - DNA, 20 bp, synthetic construct
  source 1..20: mol_type = other DNA
aaacgacggcaatggtgttg

SEQ ID NO: 14 - DNA, 20 bp, synthetic construct
  source 1..20: mol_type = other DNA
cggcggagataagagatccg

SEQ ID NO: 15 - DNA, 20 bp, synthetic construct
  source 1..20: mol_type = other DNA
acctaagagccgctttgtcg

SEQ ID NO: 16 - DNA, 20 bp, synthetic construct
  source 1..20: mol_type = other DNA
tgggtttagctctggttccg

SEQ ID NO: 17 - DNA, 20 bp, synthetic construct
  source 1..20: mol_type = other DNA
atccgagcgcctttcttgtt

SEQ ID NO: 18 - DNA, 20 bp, synthetic construct
  source 1..20: mol_type = other DNA
tcggtgagaagcacagggtg

SEQ ID NO: 19 - DNA, 20 bp, synthetic construct
  source 1..20: mol_type = other DNA
ggttgggatgggtcagaagg

SEQ ID NO: 20 - DNA, 39 bp, synthetic construct
  source 1..39: mol_type = other DNA
cgacgacaagaccgtcatgtgcttactcaacaaggtggc

SEQ ID NO: 21 - DNA, 38 bp, synthetic construct
  source 1..38: mol_type = other DNA
gaggagaagagccgtcgtgaacctgtggatgaaggatc

SEQ ID NO: 22 - DNA, 20 bp, synthetic construct
  source 1..20: mol_type = other DNA
tgtgcgtaaacgggaaaggt

SEQ ID NO: 23 - DNA, 20 bp, synthetic construct
  source 1..20: mol_type = other DNA
gcaacgtcaggttcgttcac

SEQ ID NO: 24 - DNA, 20 bp, synthetic construct
  source 1..20: mol_type = other DNA
agggagccgatgagaaggat

SEQ ID NO: 25 - DNA, 20 bp, synthetic construct
  source 1..20: mol_type = other DNA
aatgcggtcgtttgaagctc

SEQ ID NO: 26 - DNA, 20 bp, synthetic construct
  source 1..20: mol_type = other DNA
atcccagcaagtggatcgtg

SEQ ID NO: 27 - DNA, 20 bp, synthetic construct
  source 1..20: mol_type = other DNA
ggtttcgccttgcaccaaat

SEQ ID NO: 28 - DNA, 20 bp, synthetic construct
  source 1..20: mol_type = other DNA
ctgggaatggcctcaaccat

SEQ ID NO: 29 - DNA, 20 bp, synthetic construct
  source 1..20: mol_type = other DNA
catgggtgtgcattggtgac

SEQ ID NO: 30 - DNA, 21 bp, synthetic construct
  source 1..21: mol_type = other DNA
gtcaaaaatggctgcagtgac

SEQ ID NO: 31 - DNA, 21 bp, synthetic construct
  source 1..21: mol_type = other DNA
gcaacagtagaaaacgactcc

SEQ ID NO: 32 - DNA, 21 bp, synthetic construct
  source 1..21: mol_type = other DNA
gagtcaaactccaatgccaag

SEQ ID NO: 33 - DNA, 20 bp, synthetic construct
  source 1..20: mol_type = other DNA
cctccagggggtataggctt

SEQ ID NO: 34 - DNA, 21 bp, synthetic construct
  source 1..21: mol_type = other DNA
gaaggacgcatggaggagatt

SEQ ID NO: 35 - DNA, 20 bp, synthetic construct
  source 1..20: mol_type = other DNA
tccttcctcgtccacatttc

SEQ ID NO: 36 - DNA, 22 bp, synthetic construct
  source 1..22: mol_type = other DNA
tcatcatcagcagcagaacgag

SEQ ID NO: 37 - DNA, 21 bp, synthetic construct
  source 1..21: mol_type = other DNA
ccagaagctgaaatggtgtgg

SEQ ID NO: 38 - DNA, 22 bp, synthetic construct
  source 1..22: mol_type = other DNA
tcttgctaccattcctacggtc

SEQ ID NO: 39 - DNA, 20 bp, synthetic construct
  source 1..20: mol_type = other DNA
tttgaagcaaggctcttccg

SEQ ID NO: 40 - RNA, 21 nt, synthetic construct
  source 1..21: mol_type = other RNA
tattgttgttcatcttcggca

SEQ ID NO: 41 - RNA, 21 nt, synthetic construct
  source 1..21: mol_type = other RNA
ccgaagatgaacaacaatatt

SEQ ID NO: 42 - RNA, 21 nt, synthetic construct
  source 1..21: mol_type = other RNA
aaagaagaagataacaatggc

SEQ ID NO: 43 - RNA, 21 nt, synthetic construct
  source 1..21: mol_type = other RNA
cattgttatcttcttctttat

SEQ ID NO: 44 - RNA, 21 nt, synthetic construct
  source 1..21: mol_type = other RNA
aagaagaagataacaatggct

SEQ ID NO: 45 - RNA, 21 nt, synthetic construct
  source 1..21: mol_type = other RNA
ccattgttatcttcttcttta

SEQ ID NO: 46 - RNA, 21 nt, synthetic construct
  source 1..21: mol_type = other RNA
tcaatgaaatgaacactagcc

SEQ ID NO: 47 - RNA, 21 nt, synthetic construct
  source 1..21: mol_type = other RNA
ctagtgttcatttcattgagt

SEQ ID NO: 48 - RNA, 21 nt, synthetic construct
  source 1..21: mol_type = other RNA
ttagaagattggaaatgggtt

SEQ ID NO: 49 - RNA, 21 nt, synthetic construct
  source 1..21: mol_type = other RNA
cccatttccaatcttctaatc

SEQ ID NO: 50 - DNA, 20 bp, synthetic construct
  source 1..20: mol_type = other DNA
gtcgttgcaccacctgaaag

SEQ ID NO: 51 - DNA, 20 bp, synthetic construct
  source 1..20: mol_type = other DNA
tgaacgattcctggacctgc

SEQ ID NO: 52 - DNA, 20 bp, synthetic construct
  source 1..20: mol_type = other DNA
tgccgcaacattagacccat

SEQ ID NO: 53 - DNA, 20 bp, synthetic construct
  source 1..20: mol_type = other DNA
ccagctcgaaagattccggt

SEQ ID NO: 54 - DNA, 20 bp, synthetic construct
  source 1..20: mol_type = other DNA
tccggtgttggcttaccttc

SEQ ID NO: 55 - DNA, 20 bp, synthetic construct
  source 1..20: mol_type = other DNA
taatgggttccgagcggttt

SEQ ID NO: 56 - DNA, 20 bp, synthetic construct
  source 1..20: mol_type = other DNA
ccagtccatggaagaccacg

SEQ ID NO: 57 - DNA, 20 bp, synthetic construct
  source 1..20: mol_type = other DNA
cgattctccaccgtcgacat

SEQ ID NO: 58 - DNA, 20 bp, synthetic construct
  source 1..20: mol_type = other DNA
tactggcagaacgctttggt

SEQ ID NO: 59 - DNA, 20 bp, synthetic construct
  source 1..20: mol_type = other DNA
cggaggcgtcataggttctc

SEQ ID NO: 60 - DNA, 20 bp, synthetic construct
  source 1..20: mol_type = other DNA
aggccgatggtttgatgacc

SEQ ID NO: 61 - DNA, 20 bp, synthetic construct
  source 1..20: mol_type = other DNA
acgacaacctcgtcctgttc

SEQ ID NO: 62 - DNA, 20 bp, synthetic construct
  source 1..20: mol_type = other DNA
ggagtcaccagactacgacg

SEQ ID NO: 63 - DNA, 20 bp, synthetic construct
  source 1..20: mol_type = other DNA
gctgactcttaccatgccgt

SEQ ID NO: 64 - DNA, 20 bp, synthetic construct
  source 1..20: mol_type = other DNA
ttgcaaggttgtttggaccg

SEQ ID NO: 65 - DNA, 20 bp, synthetic construct
  source 1..20: mol_type = other DNA
tagggagcgttgaaggatgc

SEQ ID NO: 66 - DNA, 20 bp, synthetic construct
  source 1..20: mol_type = other DNA
taccggagctaccttgtcct

SEQ ID NO: 67 - DNA, 20 bp, synthetic construct
  source 1..20: mol_type = other DNA
cgcaacattctcatccgctg

SEQ ID NO: 68 - DNA, 20 bp, synthetic construct
  source 1..20: mol_type = other DNA
ggttatcggtgagcctgtgt

SEQ ID NO: 69 - DNA, 20 bp, synthetic construct
  source 1..20: mol_type = other DNA
accccagaaaggtgttgcat

SEQ ID NO: 70 - DNA, 2000 bp, Abelmoschus esculentus
  source 1..2000: mol_type = genomic DNA
tttttgtctgaactccgttaaggacatatctcatgacaacctctctggctctagtgtcagctctatcatttcccaccagtcaatggtgtcacccttgaataaggcagtgacgcactcgagcttaccctatggggtaaggtataactgtctgagggtgtggagagtctccctaaaccattagtctactgttatgagatcgtcatcctctgaactgaagaatttgatggtcttatatctacggagtctctctactagaaactcttgggatactacaactggctaggctggaggtgttggtgggggaggtggtgtagttggggcgctaggtatggtcctaatgatctacaaagagtttaggtgaaaaaattataaaaaatatgaagaaatatataatttatttataaattttataaattttcctaaaattatgaaaataataaaaataaaacagaaaatattttaagaattctaaatgatataaaaacaaaaaatgtacaaaagtataaaattcataaaaaaattaaaaaagtaattaccaactataagtttcttttataaaaaaattacaaaaaagtaaatttatattgtagatgagcacgtatcgaccttgcaagttgaatatttcttaattacatggtttcaacgagtttcatggggaaatgctcacgtgatgcagtcttaatatactgctaaaaaaacgttatcagttttattattgtttaaataacaaattcagtaaaaaattaccggaaacattattaagcgctaacggttttgtattattgagctttttaccgaatttggtaaaaatgaccgaaaatttttagatgctacggttttgttattgttggacacttttaccggaaatgttaaaaaattgatgttttagataaaataataatattttaggtaattttttatcaattttattatttaaacaataaccaaaccggtaacgtcttattttagtcttatattaaagtggcacactgtaaaactttcgagtttcatgacattggcaagtttcggtgcatgaagacgaaacaattatttttgtaaaaacagattttttttacctttgagcttatttttataaaagaaattaattttaattattttgtttataattttatgattttataattttgttttcatatatatatatatatatataaatataaaatatatatatattttcaattttagtaattttataaaaatatttttaataaaactttaaatatatttttataatttttcttattttcataattatttacaatagacttaataaaaatgtcatatagtcaagaattattattcatgtattataccttaaaaatatccaacttaagaaatatcgaaaaaatcacactcacatctgacttgagtagagtttaggagatctcatccaatccgaaattcgatcaaattttttggtcaaaaatcacactcaacctatatatatacagggttagaatctaattacatagtgtaaaactaaaaattatatagttttagttttacaccattttaggatcgttagatgaggggaaattaaagactgagattaacaataggaaatgcgaagacactcactagaaaaaaaataagaagttaatctcaacattgatttatctcttccaacgatattaaaagtgtaaaactaaaaaaattactataaatgtaagtgaataataactcatatatatatatatagatagataaatattaaataaaataagaatttaattaaaaaatcttattaaaattatctctaaatttataaaaaataattataatattacaccaggcttgatctgattatttatcacgatccgacccaataatacctctaaaaaacccaatctgaatccaatacgacttaagaaatatccgaactggcaattcacctaccttatctcgttgtttcccacctataaacaaacaacaggaactgaaaaaccagaagcagaagaagcaacaacagattggaagac

SEQ ID NO: 71 - DNA, 2000 bp, Abelmoschus esculentus
  source 1..2000: mol_type = genomic DNA
cctcattttctgttttacaggtagtttgagctactaattactagtctaaaatccatttcgatttgttctataaaatgtatggattaacatgtagaatttcgtattgatatcggaaatgtcaacaatttctttcacatgttataggtctcttgtctttccttgttgaagttggtttagaatcagaatgtcacgagcacgaaatgtcttggttgcttctggcttactagcttttgtcgctgcaggcttagcatttccgttttatatggcgtaagtataattcatttttcaacaacaatgtgttgttgggagctatatggttatcaactttcaaaagttcaataacggtttgtatcgtccaggacatctaggaaacctgtggattcatctaaaccacttcctccgcaggcaacttttcgagggccttacataaatactggttcacgtgatgttggacctgatcaccaaatttacccgaagaaatgatctttattttctgttgaattaactcctgttaactgtaagttaaccgtatgctcctttcgttcgtgcaccacgtgaattgactatgcatttgaaattaggctttctttccaactattgggtgcgtatgtggaggctgaactaggaattcgctttgcttcctgcagcatgatgagtattgtgtgacaactgaacatgaaaaatttattttgtaagaatttagatatgaaaaattgttatgctttctctgtggaaatcaagtgcataacatgaagaggaaaagagcagaggctatactttgttttgcttttatttttgtcatgatcttatgttaccttttattttcttttcccccgatttgtctcgtgttcttatttctaacttaagatgaaatataagcatcatctattaaaatgatgtcgagtagtttacgcttccgacaggcgtgactgtgttcgatatgttacggccgagacttgttcatggtagtaggttctcatctaattgattcggccgtaaaagatttcttctaagtttcttgataaagcccaggatttgttaatttattgctttgtggttggggctgcctgcagttcttgcaacagagcatacctttcttgtatctttatacagggtgagggtggctctggccctctttttggtttaagaacttcttagatcattgttaaattaggcaaattgagcactgaatttatgcaaataggccctcatcatcttttttaacactttatgttacagtatctgacgtgtggccttctttctttattcctccgaatttcatcaactctttcgttctttcttttcccaattttcttagtaaccaaacagaaaatggaagaaaaatagatggatataataaaacataatccttttaccatcttcgtagtccatttggttttcgtttaagccatcatcttgttcaatatattcttcttcgttcttgttttttaaattatttttattgaaaaatcataaatctcaaatactttagttttaaaaattcgtctattaaaaaaaatcataataatggataataattttaattttatttatttaatttgagaaattctaagtctaaaagaagttacaaaatcgaagaattcataatagtggaaaatgttttttcaacgggcatagcaaattaacttaaaataaatgtttaaaagtaaaaatatatatatttaaatatttattttactattaaatttttatataaaaaaaaaaatatgtcaagtagataattaatcacacgcgctgaaatgaaaagcttacgtgtgaaatcattccagctattattgccacgttgagtgtgaatacaagcattagttttccattgtcctttctagtttttcctggtggccaagacgagttcaattcttgcactcaactcaagttttttttttcttcttcatctttcatttttctttctaatacaagaacccagcttcagtttcttaaggagttccaaaagaagtaaataaatcagaaaaagaaaaaaa

SEQ ID NO: 72 - DNA, 2000 bp, Abelmoschus esculentus
  source 1..2000: mol_type = genomic DNA
ttaaaacttaaacgggataccaaaataaaacaagaactaaagttttaatgtcggtactaagatgaaacaaaaactaaactttaaatatgatcttgaaaaattaaaagtttaagtaccaaaacaaaccaaaattaacagtttaagaactgaactcgtaattgaccgaaataataattccaaaatatagaggacaaatctgacaatattagcaccttttccctgttaatgctggaaactaccacatcggtacatatttttgtcacctgtaaatttccatatttgaaagttaaaaccagcagtatatttttactcaacattatagacatttccttggattggattgattttaaatataaaaattatataattaataagtcgatgaggatcgaatataatattttatgttatatgatacgaacttgtaacattttaaaaatgtgttatatcatcaatctaatatataaagttatataaattatatgagaatttaaaatcgtgtaaaatcgagatcctaatcgaacatcgtttcgatctatccgaattcgataaagactcaagtgagataataaaaatgaggaagaatatctgggtgtgatggaaagcacataaaaaatattaaaatatataataattttaaattttcagaggacgactgtctcgtttatctatttgaatccttcctacaaaacatccttcaacttgcttcattagtctcaaagctctcaactggtattattaaacccctttgatcctctatttataatcattagttttcttagatagataatcatgaattattttcttaggaagatcctaaaccaataaggtggtgtttggttcaaggattgtgggattcccatgggggagaatctcacattcccatgtttggttggccggaaatctcattcccagtaatgttttattaccaatagtagattttttggggtatattatcaggaagtcattacctccattcccttaggtattgtaacttacggcgtttggactaaaatgcccttaatttttttttcaaaaatatcattcttctgtcattgaataataaaaattatacgaaagttaaattacaaatttatccttaaattattttagatttacaaaaaaaatctataattaaaattttatatgaagttcaggggttaatttttttaacctttcattaaacattttttatatttttaattttgtaaaaaaatatataaatattatgaatttaattttttaaataaaattcataaatattttacaatttgtatttatacaaatatatataaggacatttggtcaaatatcattccaataatctttattcctttaaccaaacacggtaatcttacattcctgctaaaccaaccaaacgcaaaatacaacattcccagtaatcctattctctggtaatcttttattcccactggtaatcttacattccgtgaaccaaacgcaccctaaagggtaaactaattatttttagtctaatttttttaaaattatacttaaaattatattttaattatctgttaaaaaatttaacgataatataatatgtttaaaaaaattatattaaaaaataataaaaatactatcaatatcacgttaatttctaacagtagatgattaaaataaattgattttaacaatgaatgatcgaaacataaattaaataatatctgagtgaccaaccatatataataaaagcctgaaaatataaggcgtaagagcgtcacctctttcaaaccagcacgacaacgaaaatagcgacacaaaacgccacgtgtcgacctaaacgacagcagattaggttctgctcaacgtgtggtcatcacgctcgtctgcctcgaaaggcaaattttaacaggagaaaataatcatcggaaggaaggttctttttaattctttccttgccgataaagttggagaagaagaagaataagtactgtttaaagattcgatttttccccttttagtttcaatctccgccaaaa

SEQ ID NO: 73 - DNA, 44 bp, synthetic construct
  source 1..44: mol_type = other DNA
cgctctagaactagtggatccatgtgcttactcaacaaggtggc

SEQ ID NO: 74 - DNA, 45 bp, synthetic construct
  source 1..45: mol_type = other DNA
gtcgacggtatcgataagctttcatgaacctgtggatgaaggatc

SEQ ID NO: 75 - DNA, 50 bp, synthetic construct
  source 1..50: mol_type = other DNA
ttcctgcagcccgggggatccgataaagttactgcaatttgaagaaattt

SEQ ID NO: 76 - DNA, 48 bp, synthetic construct
  source 1..48: mol_type = other DNA
tgtttttggcgtcttccatgggttttgtaatctgttcttgatgcttct

SEQ ID NO: 77 - DNA, 49 bp, synthetic construct
  source 1..49: mol_type = other DNA
ttcctgcagcccgggggatcctctttattttctgttgaattaactcctg

SEQ ID NO: 78 - DNA, 49 bp, synthetic construct
  source 1..49: mol_type = other DNA
tgtttttggcgtcttccatggctgatttatttacttcttttggaactcc

SEQ ID NO: 79 - DNA, 47 bp, synthetic construct
  source 1..47: mol_type = other DNA
tcctgcagcccgggggatccaatattagcaccttttccctgttaatg

SEQ ID NO: 80 - DNA, 47 bp, synthetic construct
  source 1..47: mol_type = other DNA
tgtttttggcgtcttccatggagattgaaactaaaaggggaaaaatc

SEQ ID NO: 81 - DNA, 489 bp, Abelmoschus esculentus
  source 1..489: mol_type = genomic DNA
atgcattaccgaggtgttagacaaaggccatgggggaagtgggcggcggagataagagatccgatcaaggccgctcgcgtctggctcggaaccttcactacagcggaagcggctgcattggcttacgacaaagcggctcttaggtttaaaggaagcaaagcaaagctaaacttccctgaaagagttcatcgaacatccgaatccggttgtctaacaactagccttgaagtcggaccggaggcgccgccgacactttcacatcccaatatatccgatcagtacgcacgagcacaggcagagcttctagccggtggaagcaatggtttaaactattgtttaccaggtggctgtggtggagcagtcggaacaccctttacttcacactccacaaccctatcttcatcttcatcgacgttaacatcttcatctgcttacggtggtttatctccgacaccgagttcggatcctccgaagcataagagagactag

SEQ ID NO: 82 - DNA, 699 bp, Abelmoschus esculentus
  source 1..699: mol_type = genomic DNA
atggggttttctgaagaaacatctgctcagttggaattcataaggagacatctctttaatgattttgtttccatggaaaactttatggtttccaatgttgataccacgaatcggagaaagcaatcgacgctcagccaacgtcgtccttgtatcaacgttatgatccctccggcgaacgttaacgtgggtttagctctggttccggttccggttgaaacgtgcgaggaaaggcattacaggggagtgagacgacgggcgtggggaaagtttgcatctgaaatcagagacccgaacaagaaaggcgctcggatttggctcggaaccttcgacaccgccattgaagctgcaaaagcgtacgacagagctgcgtttaagctacggggtagtaaagccatattgaatttccctcttgaagctggaaaattgaattcctcgtcatctgaggataattccttttccaggaaaaggagaagaagtgaagaggaggaagaaaagagagtggagttggagtgtaaagtagtgaaaagggaggagccgatggcggaggcccccaaggaggcggtggcggaaagtgtaacgccgtcgacttggacgggtttgggggataacgaagatatgaacgaaatatttagtggtcctttattatctccattatcgttacacccatggtttgggtattctaggcttgaggttatgtaa

(19) United States
(12) Patent Application Publication
(10) Pub. No.: US 2026/0275367 A1
(43) Pub. Date: Sep. 17, 2026

(54) METHOD FOR REDUCING CHLOROPHYLL DEGRADATION IN A PLANT

(71) Applicant: Zhejiang Wanli University, Ningbo (CN)

(72) Inventors: Saisai LI, Ningbo (CN); Qian WANG, Ningbo (CN); Jiahao LI, Ningbo (CN); Yaying XU, Ningbo (CN); Liyu SHI, Ningbo (CN); Wei WU, Ningbo (CN); Wei CHEN, Ningbo (CN); Zhenfeng YANG, Ningbo (CN)

(21) Appl. No.: 19/406,177

(22) Filed: Dec. 2, 2025

(30) Foreign Application Priority Data
Mar. 11, 2025 (CN) 202510284107.1

(51) Int. Cl. C12N 15/82; C12N 15/113

(52) U.S. Cl. CPC C12N 15/8218; C12N 15/1133; C12N 15/8242; C12N 2310/14

(57) ABSTRACT

A gene regulating chlorophyll degradation and coloration in okra and its applications, in the field of fruit and vegetable preservation. Through transcriptome data analysis of okra with gibberellic acid (GA) treatment, an ERF (Ethylene Response Factors) transcription factor, AeABR1 was identified. Subsequently, by constructing transiently expressed okra and transgenic Arabidopsis thaliana lines, it was confirmed that the AeABR1 gene positively regulates chlorophyll degradation in okra. Real-time quantitative fluorescence PCR (RT-qPCR) and dual luciferase assays demonstrated that AeABR1 protein reduces chlorophyll content of okra by down-regulating the expression of chlorophyll degradation gene AeCLH1, thereby participating in post-harvest color regulation. This provides preliminary insights into the molecular mechanism of AeABR1, offering a novel target gene and theoretical foundation for post-harvest preservation of okra.

### CROSS-REFERENCE TO RELATED APPLICATIONS

The present application claims the priority of a Chinese prior application No. 202510284107.1, filed on Mar. 11, 2025, and all contents of the prior application, including but not limited to the specification, drawings, claims and abstract, are incorporated in their entirety as a part of the present application.

### SEQUENCE LISTING

A sequence listing is being submitted with this application. The content of the electronic sequence listing (SEQUENCE_LISTING.xml; Size: 94208 bytes; and Date of Creation: Dec. 2, 2025) is herein incorporated by reference in its entirety.

### BACKGROUND OF THE INVENTION

### Field of the Invention

The present invention belongs to the field of fruit and vegetable preservation, and specifically relates to a gene regulating chlorophyll degradation and coloration in okra and its application.

### Description of the Related Art

Okra (Abelmoschus esculentus L. Moench) is a vegetable belonging to the Malvaceae family, native to India. Its tender pods are the primary edible part, and it is now widely cultivated in tropical and subtropical regions such as Zhejiang, Fujian, and Hainan Province. Compared to common vegetables, okra pods are rich in protein and mucilaginous polysaccharides, offering health benefits such as aiding digestion, combating fatigue, and boosting immunity, earning it the title ‘King of Vegetables’. Okra has a high water content and a large surface area, with well-developed stomata, and it is mainly harvested during the high-temperature season. After harvesting, it is prone to water loss and accelerated respiration, which leads to greening loss and ageing of the fruit, seriously affecting its edibility and commercial value. Fruit greening caused by chlorophyll metabolism is one of the most prominent characteristics during okra's post-harvest storage, constituting a major constraint on its storage, transportation, and marketability.

The maturation and senescence of fruits and vegetables are regulated by multiple environmental factors and hormones. Increasing researches indicate that the effects of plant hormones depend on specific hormone combinations rather than the independent activity of individual hormones. During post-harvest maturation and senescence, identifying key factors in chlorophyll metabolism regulated by cross-interactions among different plant hormone signaling molecules holds significant industrial importance for maintaining the color quality of fruits and vegetables after harvest.

Transcription factors (TFs) are a class of proteins located within the cell nucleus that can specifically interact with cis-acting elements in the non-coding regions of genes, thereby directly regulating the expression of target genes. Researches on the regulation of chlorophyll metabolism pathways by transcription factors have been extensively conducted. Arabidopsis thaliana ABF2/3/4 TFs regulate abscisic acid (ABA)-mediated chlorophyll metabolism and leaf senescence by binding to the NYE1 gene promoter sequence; Arabidopsis thaliana MYC2/3/4 proteins and NAC family members ANAC019/055/072 induce jasmonic acid (JA)-mediated chlorophyll metabolism by up-regulating the expression of PAO, NYC1, and SGRI genes. The transcription factors AtERF17, SlERF16, and CitERF13 activate the PPH gene, promoting chlorophyll metabolism in Arabidopsis thaliana, tomato, and sweet orange fruits. However, current research on transcription factor involvement in chlorophyll metabolism has primarily focused on model plants like Arabidopsis thaliana. Key factors integrating different plant hormone signals to regulate post-harvest chlorophyll metabolism in fruits and vegetables remain largely undiscovered.

### BRIEF SUMMARY OF THE INVENTION

To address the above shortcomings, the present invention provides a gene for regulating chlorophyll degradation and coloration in okra leaves and its applications, belonging to the field of fruit and vegetable preservation.

This invention identified a novel ERF transcription factor, AeABR1 (the protein encoded by AeABR1 gene), by transcriptome data analysis of okra treated with gibberellic acid (GA). Subsequently, transiently expressed okra and Arabidopsis thaliana transgenic lines were constructed to confirm that the AeABR1 gene positively regulates chlorophyll degradation in okra. Furthermore, RT-qPCR and dual luciferase assays demonstrated that the AeABR1 protein reduced okra chlorophyll content by inducing the expression of the chlorophyll degradation gene AeCLH1, thereby participating in the regulation of okra color post-harvest. In summary, this study provides preliminary insights into the molecular mechanism of AeABR1, offering a novel target gene and theoretical foundation for post-harvest preservation of okra.

To achieve the aforementioned objectives, the technical solution employed by the present invention is as follows:

On the one hand, the present invention provides a method for regulating chlorophyll degradation and/or plant coloration and/or plant preservation, wherein the method comprises reducing or diminishing the ability of the AeABR1 gene to encode the AeABR1 protein, thereby decreasing the expression of the AeABR1 protein. The AeABR1 gene comprises the nucleotide sequence shown in sequence listing SEQ ID NO. 1, wherein the AeABR1 protein comprises the amino acid residue sequence shown in sequence listing SEQ ID NO. 2.

This invention investigates the function of the AeABR1 gene, demonstrating its role in the chlorophyll degradation pathway. Plant coloration refers to the phenomenon where plants exhibit different colors within the visible light spectrum through the interaction of specific pigments (such as chlorophyll) within their tissues or organs, cellular structures, and environmental factors. Therefore, this gene can also be utilized to regulate plant coloration. Furthermore, plant preservation refers to technical measures that utilize physical, chemical, or biological methods to delay the aging, wilting, or decay processes of plant organs-such as cut flowers, leaves, fruits, and vegetables—thereby maintaining their physiological activity, morphological integrity, vibrant color, and functional quality. The core objective is to extend the ornamental period, edible value, or commercial lifespan of plants after separation from the mother plant or harvest. As the subject of this invention is the fruit of okra, the primary approach involves regulating the expression of the AeABR1 gene and/or AeABR1 protein to maintain the green color of okra fruit for an extended period post-harvest. This regulation primarily involves reducing AeABR1 gene expression, thereby decreasing AeABR1 protein greening. This, in turn, lowers the transcriptional level of the chlorophyll degradation gene AeCLH1, allowing chlorophyll to remain undegraded or degrade more slowly. Ultimately, this prolongs the period during which okra fruits maintain their green color.

It is important to understand that proteins serve as the mediators through which genes exert their functions. Specifically, the AeABR1 gene regulates chlorophyll degradation because it encodes an ERF transcription factor (protein). This transcription factor (AeABR1 protein) binds to the promoters of multiple chlorophyll degradation-related genes, subsequently up-regulating or down-regulating their expression. This process promotes chlorophyll degradation, thereby fulfilling the physiological function of the AeABR1 gene. Therefore, those skilled in the art can down-regulate the expression of the AeABR1 gene and/or the AeABR1 protein according to specific implementation methods. This reduction in protein expression leads to fewer proteins binding to promoters, thereby decreasing the expression of genes related to chlorophyll degradation. Ultimately, this achieves the goal of delaying chlorophyll degradation and aging.

Additionally, in certain approaches, accelerating chlorophyll degradation within plants is also necessary. When employing such methods, overexpressing the AeABR1 gene can expedite chlorophyll breakdown. The specifics are as follows:
1. Resource Recovery and Recycling: Firstly, nitrogen recovery-chlorophyll molecules contain four nitrogen atoms. Nitrogen released during degradation (via enzymes like magnesium-depleting chlorophyllase) can be transported to new tissues (e.g., seeds, young leaves) for reuse, reducing resource waste. Second, reuse of the carbon skeleton: chlorophyll degradation products (e.g., chlorophyll esters) can be converted into other metabolites (e.g., plant hormone precursors) to participate in new tissue construction;
2. Prevent phototoxic damage: Retained chlorophyll in senescent tissues generates reactive oxygen species (ROS) under intense light, causing cellular damage. Chlorophyll degradation reduces oxidative stress. It synchronizes with the disassembly of photosystem protein complexes, preventing dysfunctional pigments from triggering photodamage.
3. Promoting tissue remodeling: Chlorophyll degradation during cereal grain maturation prevents residual greenness from affecting seed dormancy and germination traits;
4. Agricultural and food processing: Enhancing product quality: The “kill green” step in tea processing accelerates chlorophyll degradation to achieve specific color and flavor profiles (e.g., green tea retains partial chlorophyll while black tea undergoes complete degradation) Moderate chlorophyll degradation (e.g., banana ripening) improves texture and marketability.

Therefore, upregulating AeABR1 gene and/or AeABR1 protein expression levels can achieve the aforementioned objectives.

On the other hand, the present invention provides the use of AeABR1 for preparing formulations that regulate the expression of genes related to chlorophyll degradation. Said AeABR1 comprises the AeABR1 gene and/or the AeABR1 protein, wherein the AeABR1 gene comprises the nucleotide sequence shown in sequence listing SEQ ID NO. 1, and the AeABR1 protein comprises the amino acid residue sequence as shown in sequence listing SEQ ID NO. 2. Herein, regulation may be either up-regulation or down-regulation.

Furthermore, the chlorophyll degradation-related gene is any one or more of the AeCLH1, AeCLH2, AeNOL, AeSGR, AeNYC1, AeHCAR, AeRCCR, AePAO2, and AePAO4 genes.

Furthermore, the AeCLH1 gene comprises the nucleotide sequence shown in SEQ ID NO. 3; the AeCLH2 gene comprises the nucleotide sequence shown in SEQ ID NO. 4; the AeNOL gene comprises the nucleotide sequence shown in SEQ ID NO. 5; the AeSGR gene comprises the nucleotide sequence shown in SEQ ID NO. 6; the AeNYC1 gene comprises the nucleotide sequence shown in SEQ ID NO. 7; the AeHCAR gene comprises the nucleotide sequence shown in SEQ ID NO. 8; The AeRCCR gene comprises the nucleotide sequence shown in SEQ ID NO. 9; The AePAO2 gene comprises the nucleotide sequence shown in SEQ ID NO. 10; The AePAO4 gene comprises the nucleotide sequence shown in SEQ ID NO. 11.

Furthermore, the chlorophyll degradation-related gene is AeCLH1, wherein the AeCLH1 gene comprises the nucleotide sequence shown in sequence listing SEQ ID NO. 3.

After confirming the physiological function of the AeABR1 gene, this invention employed RT-qPCR assays to detect mRNA expression levels of multiple chlorophyll degradation genes in okra lines overexpressing the AeABR1 gene. Results revealed significantly increased expression levels of AeCLH1, AeNOL, AeSGR, AeNYC1, AeRCCR, AePAO2, and AePAO4 were significantly increased. Further analysis of gene promoters suggested that AeABR1 protein may directly regulate the expression of AeCLH1, AePAO, and AeNYC1 genes (ERF transcription factor binding motifs (DRE motifs) were found in the promoters of those genes). This was validated using a dual luciferase reporter system, revealing that AeABR1 protein directly binds only to the AeCLH1 gene promoter and upregulates its expression. Conversely, reducing AeABR1 protein expression decreased its binding to the AeCLH1 gene promoter, thereby lowering AeCLH1 gene expression.

On the other hand, the invention provides an siRNA for use in preparing formulations that regulate chlorophyll degradation and or plant coloration and/or plant preservation, characterized in that said siRNA (siAeABR1s) targets the AeABR1 gene, wherein the AeABR1 gene comprises the nucleotide sequence shown in SEQ ID NO. 1.

Furthermore, said siRNA comprises any one or more pairs of nucleotide sequences shown in SEQ ID NO. 40~SEQ ID NO. 41, SEQ ID NO. 42~SEQ ID NO. 43, SEQ ID NO. 44~SEQ ID NO. 45, SEQ ID NO. 46~SEQ ID NO. 47, SEQ ID NO. 48~SEQ ID NO. 49. Preferably, SEQ ID NO. 40~SEQ ID NO. 41, SEQ ID NO. 42~SEQ ID NO. 43, and SEQ ID NO. 44~SEQ ID NO. 45.

Furthermore, the siRNA comprises the nucleotide sequence shown in SEQ ID NO. 42~SEQ ID NO. 43 of the sequence list.

Five pairs of siRNAs were designed targeting the AeABR1 gene, among which only three pairs significantly decreased AeABR1 gene expression level. Okra leaves treated with siAeABR1-2 (SEQ ID NO. 42~SEQ ID NO. 43) exhibited deeper leaf coloration and maintained greener leaves for a longer period after harvest. Therefore, this siRNA is preferred for okra preservation.

Additionally, the present invention provides a method for regulating chlorophyll degradation and or plant coloration and/or plant preservation. This method involves targeting the silencing and or knockout of the AeABR1 gene, which comprises the nucleotide sequence shown in SEQ ID NO. 1.

Furthermore, the siRNA described above is used to target and silence the AeABR1 gene.

The beneficial effects of the present invention include:
1. The present invention preliminarily explores the molecular mechanism of the AeABR1 gene, providing a new target gene and theoretical basis for post-harvest okra preservation;
2. Multiple pairs of siRNAs targeting the AeABR1 gene were designed, from which the most effective pair was selected for application in post-harvest okra preservation.

### BRIEF DESCRIPTION OF THE DRAWINGS

To more clearly illustrate the technical solutions in the embodiments of the present invention or the prior art, the accompanying drawings used in the description of the embodiments or prior art will be briefly introduced below. It is evident that the drawings described below are merely some embodiments of the present invention. For those skilled in the art, other drawings may be obtained based on these drawings without creative labor.

FIG. 1A-1C: Effects of GA treatment on postharvest okra. FIG. 1A shows the phenotypic characteristics of okra stored for different durations after GA treatment; FIG. 1B: Ethylene release levels of GA-treated okra (n=3) stored for different durations; FIG. 1C: Expression levels of the AeABR1 gene in GA-treated okra (n=3) stored for different durations; ** indicates p-value<0.01, *** indicates p-value<0.001;

FIG. 2: Promoter analysis of genes associated with chlorophyll degradation in okra;

FIG. 3: Correlation analysis between okra AeABR1 gene expression levels and ethylene release, chlorophyll content; where ABR1 denotes okra AeABR1 gene expression, ETH represents okra ethylene release, and Tchl, Tchlₐ, Tchl_{b} indicate total chlorophyll, chlorophyll a, and chlorophyll b content, respectively;

FIG. 4A-4D: Subcellular localization and transient expression analysis of AeABR1; FIG. 4A shows the subcellular localization of AeABR1 protein; FIG. 4B presents phenotypic observations of okra leaves transiently overexpressing AeABR1 (n=3); FIG. 4C: Chlorophyll content measurement in okra leaves transiently overexpressing the AeABR1 gene (n=3); FIG. 4D: Effect of transient AeABR1 overexpression on the expression levels of chlorophyll degradation-related genes (CCGs). * indicates p-value<0.05, *** indicates p-value<0.001;

FIG. 5A-5C: FIG. 5A shows phenotypes ofArabidopsis thaliana lines overexpressing AeABR1 (n=3); FIG. 5B shows chlorophyll content results; FIG. 5C shows CCGs expression analysis results; ** indicates p-value<0.01, ***indicates p-value<0.001;

FIG. 6: AeABR1 protein induces expression of the chlorophyll degradation gene AeCLH1 in okra; n=3, * indicates p-value<0.05.

### DETAILED DESCRIPTION OF THE INVENTION

The preferred embodiments of the present invention will be described in further detail below in conjunction with the accompanying drawings. It should be noted that the following embodiments are intended to facilitate the understanding of the present invention and are not intended to limit it in any way. All features disclosed in the embodiments of the present invention, or all steps of the disclosed methods or processes except mutually exclusive features and/or steps, can be combined in any way.

Unless otherwise specified, the experimental methods used in the following embodiments are conventional methods; unless otherwise specified, the materials and reagents used are commercially available reagents and materials.

The specialized terminology used in this invention and their definitions are as follows:
1. Transcription factor: A class of proteins located within the cell nucleus that can specifically interact with cis-acting elements in the non-coding regions of genes, thereby directly regulating the expression of target genes.
2. Abscisic acid (ABA): An important plant hormone primarily involved in regulating plant growth and development, as well as responses to abiotic stresses such as drought, cold, and salt stress.
3. Gibberellic acid (GA): A plant hormone that promotes stem elongation, induces bolting and flowering in long-day plants under short-day conditions, accelerates seed germination, and facilitates parthenocarpy.
4. ERF transcription factors: A class of transcription factors unique to plants, involved in signal transduction processes governing growth, development, and physiological/biochemical responses.

### Example 1: Identification of the AeABR1 Gene

Previous studies have demonstrated that gibberellic acid (GA) can effectively mitigate post-harvest browning in fruits and vegetables. Therefore, this implementation first verified the effect of GA on post-harvest okra (Abelmoschus esculentus), following these specific steps:

### 1.1 GA Treatment

Okra was harvested from local farms in Ningbo. Fruits were selected based on uniform size, color, absence of mechanical damage, consistent relative maturity, no visible disease, commercial harvest stage, and length of 10-12 cm. They were randomly divided into two groups (treatment and control), each containing 150 fruits. These 150 fruits were further subdivided into three subgroups, serving as three biological replicates for each treatment. The treatment group underwent the following procedure: okra was immersed in a 10 g·L⁻¹ GA solution (purchased from Shanghai Aladdin Company, purity≥95%) for 10 minutes. The control group was immersed in distilled water for 10 minutes.

The treated okra was air-dried naturally (to remove surface moisture) and packaged in 0.03 mm polyethylene bags, with 10 okra pods per bag. The okra was transferred to a constant-temperature chamber maintained at 90% relative humidity and 10° C. for 12 days of storage. Samples were collected at 0, 3, 6, 9, and 12 days. Each sampling point comprised 3 biological replicates, with 10 okra pods per replicate. First, measure ethylene emission from the okra pods. Then, remove seeds, cut the pods into small pieces, flash-freeze them in liquid nitrogen, and store long-term at −80° C.

### 1.2 Ethylene Release Measurement

Ten okra fruits were randomly selected and placed in a 1.5 μL sealed gas collection vessel for 3 hours. The headspace gas sample was collected and introduced through a rubber membrane into a gas chromatograph (GC-2014C, Shimadzu, Japan) equipped with a flame ionization detector (FID). The GC parameters were set as follows: 30 m×0.32 mm×0.25 m column (RTx-5MS, Shimadzu, Japan), column temperature 80° C., injector temperature 230° C., detector temperature 270° C., injection volume 1 mL. Calculate ethylene release rates during post-harvest storage of fruits, expressed as mL·kg⁻¹·h⁻¹. The calculation formula is:

$$Ethylene release rate ( mL \cdot {kg}^{- 1} \cdot {h}^{- 1} ) = c \times V \times {m}^{- 1} \times {t}^{- 1} \times {10}^{- 3}$$

In the formula: c represents the ethylene content in the sample gas measured by gas chromatography, μL/L; V denotes the volume of the sealed container (L); m indicates the sample mass (kg); t signifies the measurement time (h).

### 1.3 Chlorophyll Content Determination

Prepare an extraction solution by mixing acetone and anhydrous ethanol in a 1:1 (v/v) ratio. Weigh 5 g of ground okra fruit sample and mix thoroughly with 10 mL of the acetone-ethanol (1:1) solution. Place the mixture on ice in the dark for 30 min to allow complete extraction. Centrifuge at 4° C. and 12,000 g for 10 min. Use the mixed extraction solution as a blank for zero adjustment. Take 1 mL of the supernatant and measure its absorbance at 663 nm and 645 nm using a UV spectrophotometer. Calculate the chlorophyll content in the sample, expressed in mg·kg⁻¹. Calculation formulas:

$${Chl}_{a} = ( 12.7 {D}_{663} - 2.59 {D}_{645} ) \times V / W ;$$

$${Chl}_{b} = ( 22.9 {D}_{645} - 4.67 {D}_{663} ) \times V / W ;$$

$$TChl = {Chl}_{a} + {Chl}_{b}$$

Where: Chlₐ, Chl_{b}, and TChl represent chlorophyll a, chlorophyll b, and total chlorophyll content, respectively, in mg·kg⁻¹; D₆₆₃ and D₆₄₅ denote the absorbance values of the extract at wavelengths 663 nm and 645 nm; V is the final total volume of the extract after dilution, in mL; W is the weighed sample mass, in g.

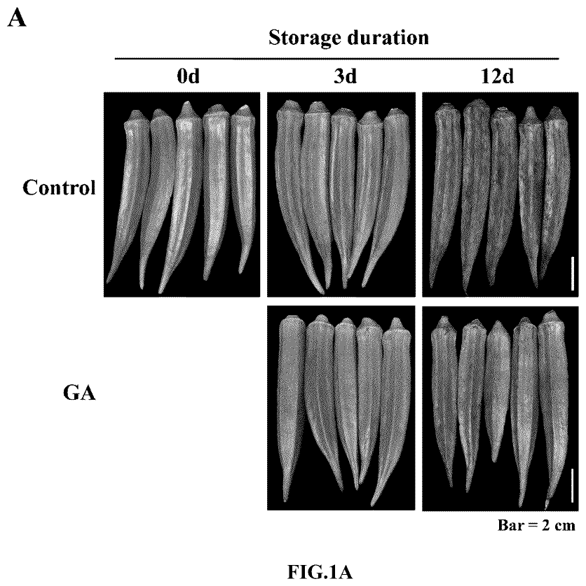

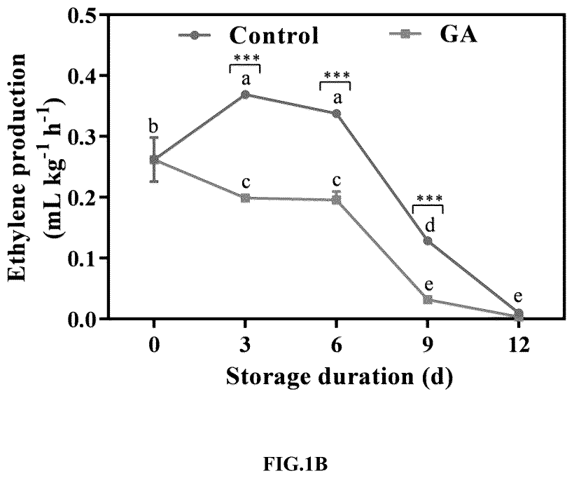

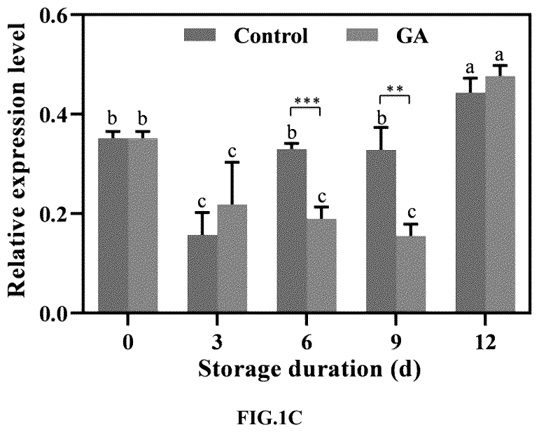

Results indicate that compared to the control, okra fruit treated with GA maintained higher chlorophyll content after 12 days, with significantly reduced browning (FIG. 1A). This demonstrates the role of GA treatment in sustaining post-harvest quality in okra.

Ethylene is a plant hormone that primarily promotes ripening and senescence processes. Excessive ethylene release from okra may accelerate its own maturation, leading to poor texture, spotting or browning, reduced storage life, and diminished quality. It may also affect the freshness of surrounding fruits and vegetables, resulting in overall cargo losses. Experimental results showed that during okra storage, ethylene release in the control group peaked on day 3 and then declined. In contrast, GA-treated okra exhibited significantly lower ethylene release than the control group on days 3, 6, and 9 (FIG. 1), consistent with the phenotypic observations in FIG. 1A.

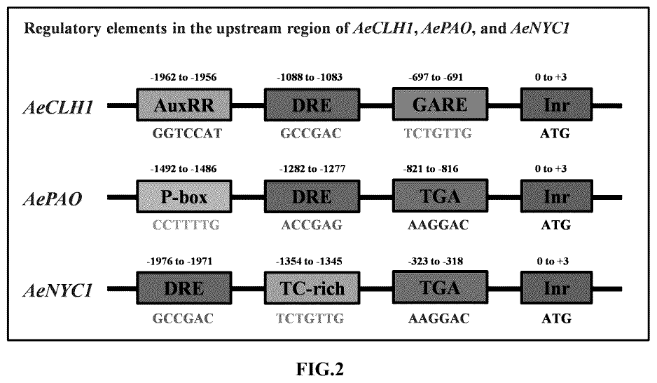

To further investigate the molecular mechanism of GA-mediated chlorophyll degradation, RNA was extracted from the aforementioned control and GA-treated okra samples stored for different durations (0 d, 3 d, 6 d, 9 d, and 12 d) (n=3) using the Polysaccharide Polyphenol Plant RNA Extraction Kit (Vazyme, RC401). The RNA samples were sent to Guangzhou Gedi Bio-Technology Co., Ltd. for transcriptome sequencing and data analysis, identifying 926 differentially expressed genes (DEGs), including 364 up-regulated genes and 562 down-regulated genes. Given the critical role of abscisic acid (ABA) in leaf chlorosis, the significant reduction in ethylene release from GA-treated okra fruits (FIG. 1), and the presence of DRE motifs (ERF transcription factor binding sites) in promoters of chlorophyll degradation-related genes (e.g., AeCLH1, AePAO, and AeNYC1) (FIG. 2), this study focused on differentially expressed genes (DEGs) associated with ethylene and ABA. Among these DEGs, potential transcription factors from the ERF family were identified: AeABR1 (ABA repressor 1), AeERF114 (SEQ ID NO.81), and AeERF105 (SEQ ID NO.82). The coding sequence (CDS) of the AeABR1 gene is shown in SEQ ID NO. 1, and its amino acid sequence is shown in SEQ ID NO. 2. The full-length AeABR1 gene spans 1245 bp, encoding a protein composed of 414 amino acid residues with a molecular weight of approximately 44.91 kDa and an isoelectric point of 6.32.

To preliminarily investigate whether AeABR1, AeERF114, and AeERF105 play roles in chlorophyll degradation in okra leaves, this example utilized BeaconDesigner7 software to design real-time quantitative PCR primer pairs for the AeABR1, AeERF114, and AeERF105 genes, respectively, corresponding to SEQ ID NO. 12~SEQ ID NO. 13, SEQ ID NO. 14~SEQ ID NO. 15, and SEQ ID NO. 16~SEQ ID NO. 17, respectively. The RNA expression levels of these genes in okra subjected to different treatments were double-verified through PCR cloning product sequencing and melting curve analysis of the internal reference gene. The real-time fluorescent quantitative PCR reaction system consisted of 20 L, including 10 μL Hieff® qPCR SYBR Green Master Mix (High Rox Plus) (Yeasen, China), 5 μL cDNA (transcribed from okra RNA extracted for transcriptome sequencing), 10 M primer pairs (SEQ ID NO. 12~SEQ ID NO. 13 or SEQ ID NO. 14~SEQ ID NO. 15 or SEQ ID NO. 16-SEQ ID NO. 17) at 0.5 μL each, and 4 μL ddH₂O. The reaction program was as follows: 95° C. pre-denaturation for 5 min; 95° C. for 15 s, 55° C. for 30 s, 75° C. for 15 s, repeated for 40 cycles. Simultaneously, the okra AeActin gene (GQ340770, primer pair SEQ ID NO. 18 and SEQ ID NO. 19) served as an internal control. The expression level of the AeABR1 gene was calculated using the relative quantitative 2^{-ΔCT} method.

Results indicated (FIG. 1C) that during the mid-to-late storage period (after 6 days of storage), AeABR1 gene expression gradually increased in okra fruits from the control group. while GA-treated okra exhibited significantly lower AeABR1 expression levels at both 6 and 9 days post-storage compared to the control group. This indicates that GA inhibits AeABR1 gene expression, suggesting that AeABR1 promotes the browning process in okra. However, GA exhibited a much weaker inhibitory effect on AeERF114 and AeERF105 gene expression compared to its suppression of AeABR1. Therefore, AeABR1 was selected as the target gene for subsequent studies.

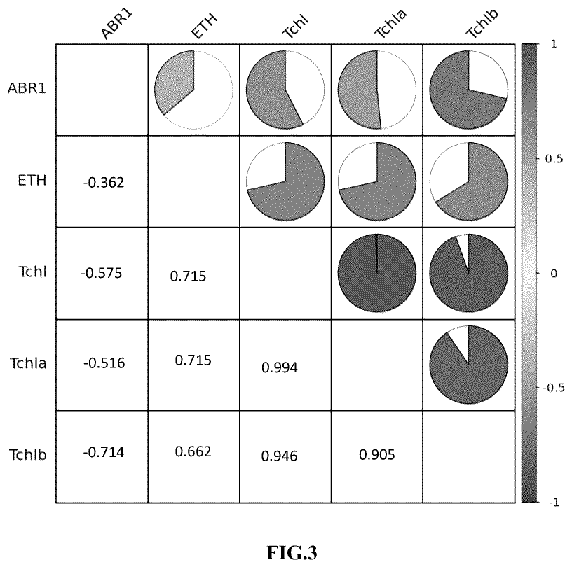

Furthermore, this example also measured chlorophyll content in okra (n=3) stored for varying durations (0 d, 3 d, 6 d, 9 d and 12 d) after treatment with distilled water or GA, following the procedure described in step “1.3.” The relationship among AeABR1 gene expression levels, ethylene release, and chlorophyll content was analyzed. Correlation analysis revealed that AeABR1 expression levels were negatively correlated with both ethylene release (R=−0.362, P<0.05) and chlorophyll content (R values ranging from −0.714 to −0.516, P<0.05) (FIG. 3), further suggesting a potential association between the AeABR1 gene and chlorophyll degradation.

### Example 2: Functional Analysis of the AeABR1 Gene Using Transient Expression Technology

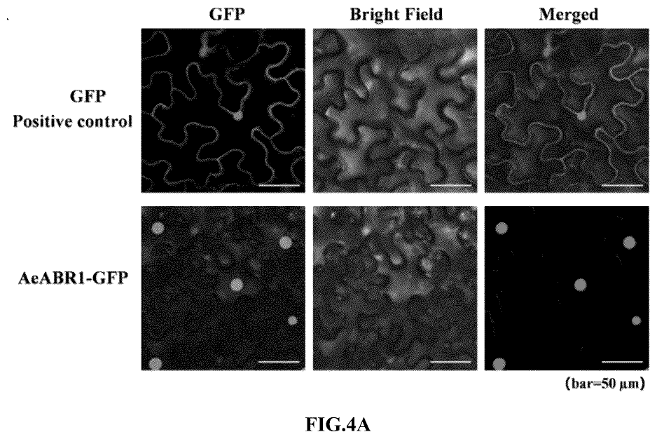

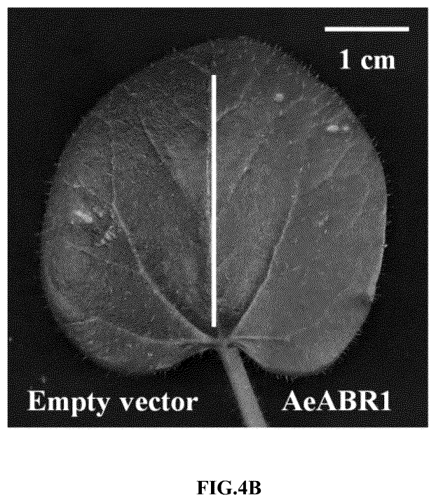

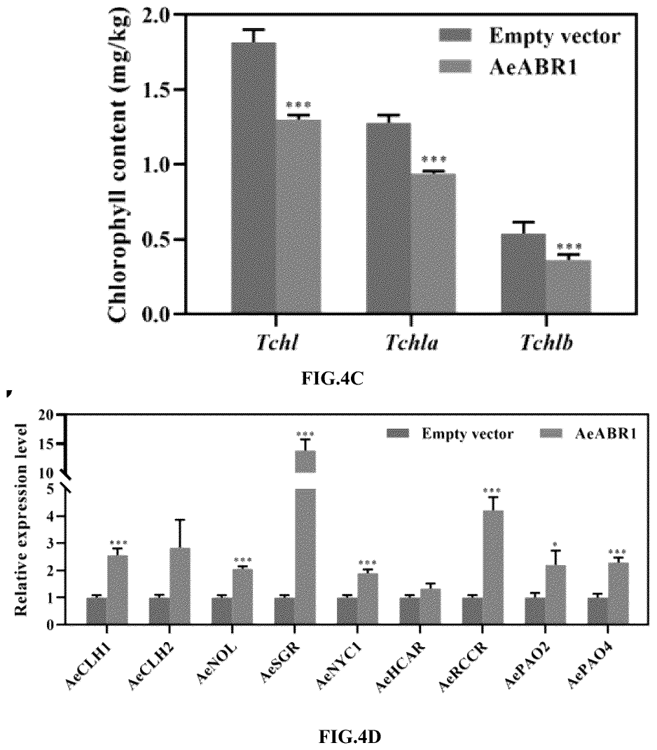

Example 1 utilized transcriptome data to screen for the AeABR1 gene associated with chlorophyll degradation in okra. This example first validated the subcellular localization of the AeABR1 protein, with the specific procedure as follows: Using amplification primers containing Lic ligation sequences, combined with SEQ ID NO.20 and SEQ ID NO.21 sequences and 2×Phanta Max Master Mix (Vazyme, P515), the full-length CDS sequence of the AeABR1 gene was amplified. The vector was constructed using Lic technology instead of traditional restriction enzyme ligation. dATP was added to the target fragment using T4 DNA polymerase (New England Biolabs, M0203). The pCV-eGFP-N1 vector (containing the Lic connection sequence) was linearized by digestion with Fast digest Apa I enzyme. After purification, T4 DNA polymerase (New England Biolabs, M0203) was used to add dTTP to the vector. The amplified AeABR1 CDS sequence (with dATP) was then ligated into the linearized pCV-eGFP-N1 vector (with dTTP) to obtain the pCV: AeABR1-eGFP dual-expression vector. Subsequently, the dual-recombinant plasmid was transformed into EHA105 strain (Shanghai Weidi Biotechnology Co., Ltd., AE1010) via the freeze-thaw method, and glycerol stocks were preserved. Agrobacterium containing pCV: AeABR1-eGFP and empty pCV-eGFP-N1 vectors were activated in two rounds at 28° C. Bacterial cells were collected by centrifugation at 5,000 rpm for 5 minutes at room temperature; Resuspend the cells in infection buffer (10 mM MgCl₂, 10 mM MES, 200 M acetosyringone, pH 5.6) to an OD₆₀₀=1.0, incubated at room temperature in the dark for 4 h; the resuspension was then injected into the 4th-5th leaves of 4-6-week-old Nicotiana benthamiana plants using a syringe, with 100 μL of bacterial suspension injected per injection; post-injection plants were cultured in an artificial climate chamber under 16 h light/8 h dark conditions at 25° C. for 60 h before observation under a laser confocal microscope; A negative control was included, where Nicotiana benthamiana leaves were infected with Agrobacterium containing the pCV-eGFP-N1 plasmid. Results showed that the GFP protein (control group) was expressed throughout the cell, including the nucleus and cell membrane, indicating successful Agrobacterium infection of Nicotiana benthamiana leaves. In contrast, the majority of AeABR1-GFP fusion protein was localized to the nucleus (FIG. 4A), consistent with the function of a transcription factor.

Subsequently, to further investigate the function of the AeABR1 gene, this example followed the aforementioned steps to introduce Agrobacterium containing the pCV:AeABR1-eGFP dual-expression vector into okra leaves, injecting 150 μL of bacterial suspension per injection. Phenotypic changes in the leaves were observed after 7 days. Simultaneously, leaf samples were collected: one portion was analyzed for chlorophyll content, while another was frozen at −80° C. for subsequent experiments, including RNA extraction, reverse transcription, and detection of expression levels of chlorophyll degradation-related genes. The quantitative real-time PCR primers for okra chlorophyll degradation-related genes are listed in Table 1. The experimental procedures for measuring chlorophyll content and detecting the expression levels of the aforementioned genes were identical to those described above.

TABLE 1
Primers for RT-qPCR of Chlorophyll Degradation-Related Genes in Okra

| Gene | | Serial | | Serial |
|---|---|---|---|---|
| Name | sense (5′-3′) | Number | antisense (5′-3′) | Number |
| AeCLH1 | TGTGCGTAAACGGGAAAGGT | SEQ | GCAACGTCAGGTTCGTTCAC | SEQ |
| | | ID | | ID |
| | | NO. 22 | | NO. 23 |
| AeCLH2 | AGGGAGCCGATGAGAAGGAT | SEQ | AATGCGGTCGTTTGAAGCTC | SEQ |
| | | ID | | ID |
| | | NO. 24 | | NO. 25 |
| AeNOL | ATCCCAGCAAGTGGATCGTG | SEQ | GGTTTCGCCTTGCACCAAAT | SEQ |
| | | ID | | ID |
| | | NO. 26 | | NO. 27 |
| AeSGR | CTGGGAATGGCCTCAACCAT | SEQ | CATGGGTGTGCATTGGTGAC | SEQ |
| | | ID | | ID |
| | | NO. 28 | | NO. 29 |
| AeNYC1 | GTCAAAAATGGCTGCAGTGA | SEQ | GCAACAGTAGAAAACGACT | SEQ |
| | C | ID | CC | ID |
| | | NO. 30 | | NO. 31 |
| AeHCAR | GAGTCAAACTCCAATGCCAA | SEQ | CCTCCAGGGGGTATAGGCTT | SEQ |
| | G | ID | | ID |
| | | NO. 32 | | NO. 33 |
| AeRCCR | GAAGGACGCATGGAGGAGAT | SEQ | TCCTTCCTCGTCCACATTTC | SEQ |
| | T | ID | | ID |
| | | NO. 34 | | NO. 35 |
| AePAO2 | TCATCATCAGCAGCAGAACG | SEQ | CCAGAAGCTGAAATGGTGT | SEQ |
| | AG | ID | GG | ID |
| | | NO. 36 | | NO. 37 |
| AePAO4 | TCTTGCTACCATTCCTACGGT | SEQ | TTTGAAGCAAGGCTCTTCCG | SEQ |
| | C | ID | | ID |
| | | NO. 38 | | NO. 39 |

Seven days after Agrobacterium infiltration, transient overexpression of the AeABR1 gene accelerated chlorosis in the right leaf compared to the left leaf control, characterized by marked yellowing (FIG. 4B). Compared to the control group, chlorophyll content in the leaf region transiently overexpressing the AeABR1 gene was significantly reduced (FIG. 4C), consistent with the phenotype observed in okra leaves. Furthermore, in leaf cells overexpressing the AeABR1 gene, mRNA expression levels of chlorophyll degradation-related genes (CCGs, including AeCLH1, AeNOL, AeSGR, AeNYC1, AeRCCR, AePAO2 and AePAO4) were significantly increased in leaves overexpressing AeABR1 (FIG. 4D). This indicates that AeABR1 promotes chlorophyll degradation in okra by directly or indirectly regulating these chlorophyll degradation-related genes. Based on the combined experimental results from Examples 1 and 2, it can be preliminarily concluded that the AeABR1 gene plays a positive regulatory role in chlorophyll degradation in okra.

To further validate the function of AeABR1 and investigate its potential application in post-harvest preservation of okra, this example designed multiple siRNAs targeting AeABR1 (siAeABR1, see Table 2, synthesized by Sangon Biotech (Shanghai) Co., Ltd.). A 50 nM concentration of these siRNAs was injected or sprayed (injection preferred in this example) to three-week-old okra leaves at a volume of 100 μL per injection or spray. After 36 hours, the silencing efficiency of siAeABR1 was assessed (RNA extraction, reverse transcription, and quantitative real-time PCR analysis of AeABR1 gene expression, n=3) and leaf phenotypes were observed, following the procedures described above. A negative control (siGFP, sense: GCCAUGAUAUAGACGUUGUTT, antisense:ACAACGUCUAUAUCAUGGCTT) was included concurrently.

TABLE 2
siRNA Targeting AeABR1 Gene Silencing

| Gene | | Serial | | Serial |
|---|---|---|---|---|
| Name | sense (5′-3′) | Number | antisense (5′-3′) | Number |
| siAeABR1- | UAUUGUUGUUCAUCUUCGGCA | SEQ ID | CCGAAGAUGAACAACAAUAUU | SEQ ID |
| 1 | | NO. 40 | | NO. 41 |
| siAeABR1- | AAAGAAGAAGAUAACAAUGGC | SEQ ID | CAUUGUUAUCUUCUUCUUUAU | SEQ ID |
| 2 | | NO. 42 | | NO. 43 |
| siAeABR1- | AAGAAGAAGAUAACAAUGGCU | SEQ ID | CCAUUGUUAUCUUCUUCUUUA | SEQ ID |
| 3 | | NO. 44 | | NO. 45 |
| siAeABR1- | UCAAUGAAAUGAACACUAGCC | SEQ ID | CUAGUGUUCAUUUCAUUGAGU | SEQ ID |
| 4 | | NO. 46 | | NO. 47 |
| siAeABR1- | UUAGAAGAUUGGAAAUGGGUU | SEQ ID | CCCAUUUCCAAUCUUCUAAUC | SEQ ID |
| 5 | | NO. 48 | | NO. 49 |

Experiments revealed that only three pairs of siRNAs (siAeABR1-1 to 3) among the five pairs of siAeABR1s effectively downregulated the expression of the target gene. Furthermore, compared to the control, okra leaves treated with siAeABR1-1-3 exhibited significantly greener coloration, with chlorophyll content reaching 1.2 to 2 times that of the control. Additionally, the duration of green color retention in ex vivo leaves was significantly prolonged. These results not only further validate the physiological function of AeABR1s but also confirm that this gene can serve as a target gene for okra preservation. Furthermore, siAeABR1-2 (SEQ ID NO.42~SEQ ID NO.43) exhibited the optimal silencing effect, with okra leaves treated with this siRNA displaying the greenest color and highest chlorophyll content. Therefore, this siRNA is preferred for post-harvest preservation of okra.

### Example 3: Functional Analysis of the AeABR1 Gene Using Stable Genetic Technology

To further elucidate the biological function of the AeABR1 gene in GA-mediated chlorophyll degradation during okra postharvest, this example established Arabidopsis thaliana transgenic lines overexpressing AeABR1 via the floral dip method. The specific steps are as follows:

3.1 Preparation of Agrobacterium Culture for Infection of Arabidopsis thaliana

The EHA105 strain containing the pCV: AeABR1-eGFP plasmid underwent two rounds of shaking activation. It was then centrifuged at 5,000 rpm at room temperature for 5 min. The bacterial cells were collected and resuspended in an infection buffer (5 g sucrose, 90 mL ddH₂O, and 50 μL Silwet L-77), where the surfactant Silwet L-77 enhances leaf uptake. Adjust the suspension to an OD₆₀₀=0.8 for subsequent infection.

3.2 Inoculation of Arabidopsis thaliana Using floral dip method:

Take 5-week-old Arabidopsis thaliana plants, remove pods, and retain unopened inflorescences. Invert the inflorescences and fully immerse them in the infection buffer for 1 minute before removal. Each plant was placed in a plastic bag for humidity maintenance, kept completely dark and laid horizontally. After 48 hours, the bag was opened and the plant stood upright. The Arabidopsis thaliana resumed normal growth. The immersion procedure was repeated after 7 days to enhance infection efficiency;

### 3.3 Screening of the Transgenic Lines Using Kanamycin:

Prepare MS solid screening medium containing 50 mg/L kanamycin but no sucrose (Phytotech Labs, USA, M519). Treat T₀ transgenic Arabidopsis thaliana seeds with vernalization at 4° C. for 2 days. Place T₀ transgenic seeds in 2 mL centrifuge tubes, disinfect with 75% ethanol for 5 min, then rinse five times with sterile water; set aside. Sow T₀ transgenic Arabidopsis thaliana seeds in a laminar flow hood by evenly dispensing sterilized seeds onto the prepared kanamycin-resistant MS selection medium using a pipette tip. Incubate at 23° C. under 16 h light/8 h dark conditions until seed germination. Once the seedlings develop their first pair of true leaves and establish roots, transfer them to a seedling medium composed of mixed nutrient soil: vermiculite: perlite (4:3:1). Cover with plastic film to maintain 100% humidity for at least 10 days. After the transplanted seedlings stabilize, open ventilation holes in the plastic cover to allow normal growth at over 80% humidity. Harvest seeds after pod formation, collecting seeds individually from each plant for drying and storage. Continue growing and screening T₁ seeds on MS solid medium containing kanamycin; seedlings exhibiting true leaves and roots on plates constitute the T₂ generation. Continue kanamycin resistance screening of T₂ seeds to obtain homozygous transgenic Arabidopsis thaliana lines until all T₃ and subsequent generations grown on resistant plates exhibit green resistance. Once stable expression is confirmed in T₃ transgenic lines, these lines are reserved for subsequent experiments. Positive lines overexpressing the AeABR1 gene were identified via quantitative real-time PCR (qPCR) using primers SEQ ID NO.12 and SEQ ID NO.13, with Arabidopsis thaliana AtActin serving as the internal control (qPCR primers: SEQ ID NO.50 and SEQ ID NO.51). All other experimental procedures followed those described in Example 1. Following resistance screening of T₀-generation Arabidopsis thaliana seeds, 14 initial Arabidopsis thaliana lines were obtained. qPCR validation confirmed 11 positive plants, from which three independent transgenic lines (OE-2,OE-8 and OE-10) exhibiting high AeABR1 gene expression and positive rates were selected for subsequent experiments. Among these, the positive strains were ranked from highest to lowest AeABR1 gene mRNA expression levels as follows: OE-2>OE-10>OE-8. The experiments included phenotypic observation of leaves from 9-week-old positive Arabidopsis thaliana seedlings, measurement of chlorophyll content, and detection of gene expression levels related to chlorophyll degradation. The primers used for detecting gene expression are shown in Table 3, with Atactin serving as the internal reference gene. The remaining steps were identical to those described in Example 1.

TABLE 3
Primers for RT-qPCR of Chlorophyll Degradation-Related Genes in
Arabidopsis thaliana

| Gene | | Serial | | Serial |
|---|---|---|---|---|
| Name | sense (5′-3′) | Number | antisense (5′-3′) | Number |
| AtCLH1 | TGCCGCAACATTAGACCCAT | SEQ ID | CCAGCTCGAAAGATTCCGGT | SEQ ID |
| | | NO. 52 | | NO. 53 |
| AtCLH2 | TCCGGTGTTGGCTTACCTTC | SEQ ID | TAATGGGTTCCGAGCGGTTT | SEQ ID |
| | | NO. 54 | | NO. 55 |
| AtRCCR | CCAGTCCATGGAAGACCACG | SEQ ID | CGATTCTCCACCGTCGACAT | SEQ ID |
| | | NO. 56 | | NO. 57 |
| AtNOL | TACTGGCAGAACGCTTTGGT | SEQ ID | CGGAGGCGTCATAGGTTCTC | SEQ ID |
| | | NO. 58 | | NO. 59 |
| AtNYC1 | AGGCCGATGGTTTGATGACC | SEQ ID | ACGACAACCTCGTCCTGTTC | SEQ ID |
| | | NO. 60 | | NO. 61 |
| AtPAO | GGAGTCACCAGACTACGACG | SEQ ID | GCTGACTCTTACCATGCCGT | SEQ ID |
| | | NO. 62 | | NO. 63 |
| AtSGR1 | TTGCAAGGTTGTTTGGACCG | SEQ ID | TAGGGAGCGTTGAAGGATGC | SEQ ID |
| | | NO. 64 | | NO. 65 |
| AtSGR2 | TACCGGAGCTACCTTGTCCT | SEQ ID | CGCAACATTCTCATCCGCTG | SEQ ID |
| | | NO. 66 | | NO. 67 |
| AtPPH | GGTTATCGGTGAGCCTGTGT | SEQ ID | ACCCCAGAAAGGTGTTGCAT | SEQ ID |
| | | NO. 68 | | NO. 69 |

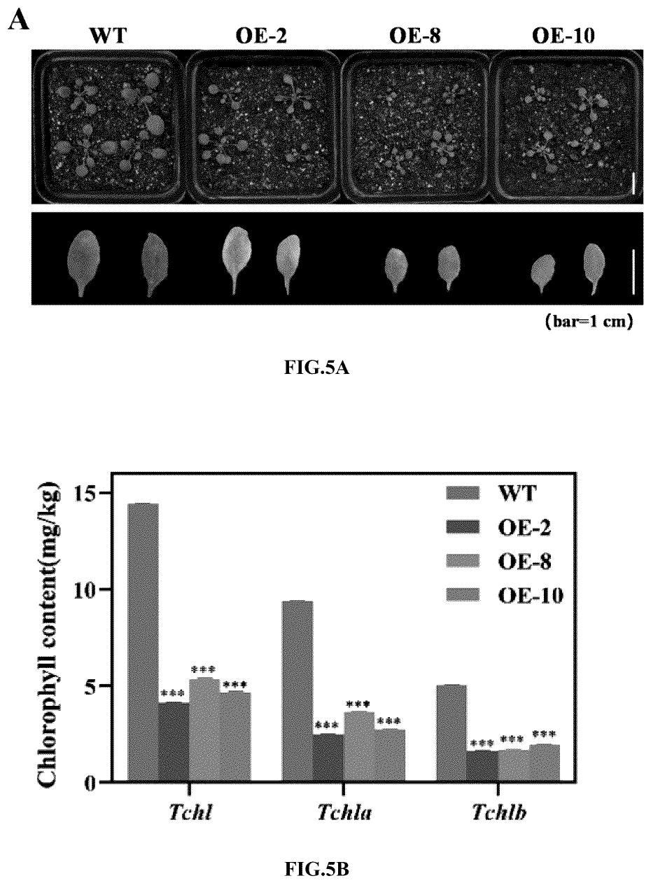

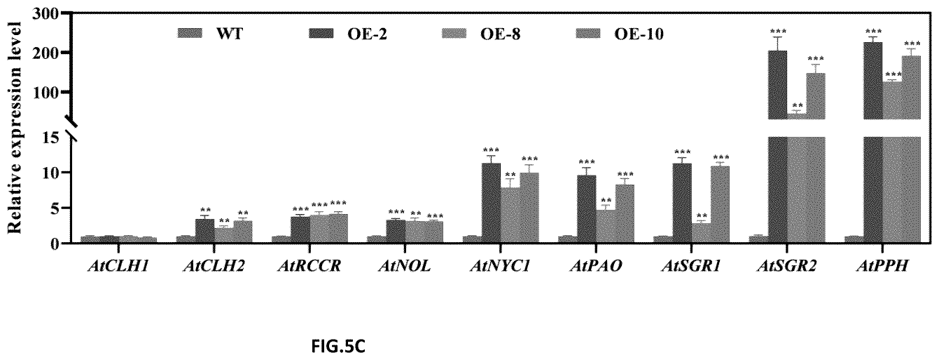

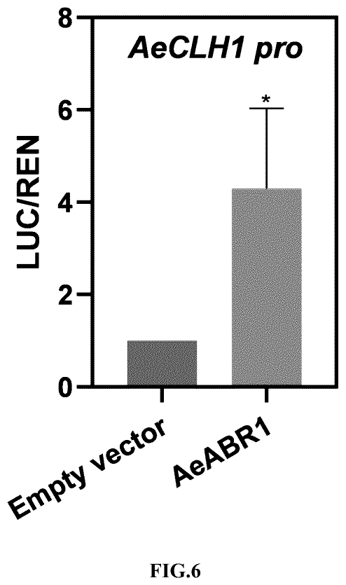

Compared to wild-type (WT) plants, Arabidopsis thaliana seedlings overexpressing the AeABR1 gene exhibited a yellowing leaf phenotype, with the OE-2 line showing the most severe yellowing due to its highest expression level of AeABR1 (FIG. 5A). Meanwhile, the chlorophyll content in leaves of three independent Arabidopsis thaliana transgenic lines overexpressing the AeABR1 gene was significantly lower than that in the control. This result corroborates the yellowing phenotype observed in the leaves of the transgenic Arabidopsis thaliana lines, with the OE-2 line exhibiting the lowest chlorophyll content. Furthermore, real-time quantitative fluorescence analysis revealed that AeABR1 overexpression significantly increased mRNA expression levels of chlorophyll degradation-related genes in Arabidopsis thaliana (such as AtCLH, AtRCCR, AtNOL, AtNYC1, AtPAO, AtSGR and AtPPH) (FIG. 5C).

In summary, the experimental results from Examples 1-3 confirm that the AeABR1 gene induces chlorophyll degradation-related genes (such as CLHR, CCR, NOL, NYC1, PAO and SGRgenes) to promote chlorophyll degradation in okra leaves and regulate its coloration. Therefore, the AeABR1 gene can serve as a target gene for post-harvest okra preservation. Among the siRNAs tested, siAeABR1-2 demonstrated the most effective comprehensive results and is thus preferred for application in okra preservation.

### Example 4: Investigation of the Molecular Mechanism of AeABR1 Gene Function

Although Examples 1-3 elucidate the physiological function of the AeABR1 gene, its molecular mechanism of action remains unknown; Simultaneously, the aforementioned examples employed qPCR to detect the RNA expression levels of multiple chlorophyll degradation genes in okra and Arabidopsis thaliana AeABR1 gene overexpression lines. Results revealed significantly upregulated expression of CLH, RCCR, NOL, NYC1, PAO and SGR genes. Subsequent analysis of these gene promoters revealed that only the promoters of AeCLH1, AePAO and AeNYC1 contained ERF transcription factor binding motifs (DRE motifs) (FIG. 2). Therefore, this embodiment employs a dual luciferase assay to investigate the regulatory relationship between transcription factor AeABR1 and the genes AeCLH1, AePAO or AeNYC1. The dual luciferase reporter system comprises firefly luciferase (F-Luc) and Renilla luciferase (R-Luc), enabling analysis of transcriptional factor effects on target gene promoters. The specific procedure is as follows:

Using homologous recombination, the full-length coding sequence of AeABR1 and the promoter sequences of AeCLH1/AePAO/AeNYC1 (SEQ ID NO. 70-72) were inserted separately into the pGreenII 0800-LUC and pGreenII 62-SK vectors. The primer sequences for constructing these vectors are shown in Table 4. The pGreenII 0800-LUC empty vector combined with pGreenII 62-SK served as a negative control. Using the freeze-thaw method, the positive recombinant plasmids were individually transformed into the GV3101 strain containing the pSoup-p19 plasmid, and glycerol stocks were preserved. The positive transformant cultures were activated twice. The bacterial suspension was adjusted to an OD₆₀₀ of approximately 0.2 using the inoculum solution and incubated at room temperature in the dark for 4 hours. Effector and Reporter recombinant vector suspensions were mixed at a 8:1 volume ratio. The resulting suspension was injected into the lower epidermis of the abaxial surface of Nicotiana benthamiana leaves. Plants were cultured in a climate chamber at 25° C. under a 16 h light/8 h dark cycle for 3 days. Excisions of 1 cm diameter from the injected region were analyzed using the Dual-Luciferase Reporter Assay System (Yeasen, China) to measure the ratio of LUC to REN luciferase activity in the leaves.

TABLE 4
Primers Required for Constructing Effector and Reporter Recombinant
Vectors

| Primer | | Serial | | Serial |
|---|---|---|---|---|
| Name | sense (5′-3′) | Number | antisense (5′-3′) | Number |
| AeABR1- | cgctctagaactagtGGATCCATGTG | SEQ ID | gtcgacggtatcgatAAGCTTTC | SEQ ID |
| pGreenII | CTTACTCAACAAGGTGGC | NO. 73 | ATGAACCTGTGGATGA | NO. 74 |
| 0800- | | | AGGATC | |
| LUC | | | | |
| AeCLH1- | ttcctgcagcccgggggatccGATAAA | SEQ ID | tgtttttggcgtcttccatggGTTTT | SEQ ID |
| pGreenII | GTTACTGCAATTTGAAGAAA | NO. 75 | GTAATCTGTTCTTGATG | NO. 76 |
| 62-SK | TTT | | CTTCT | |
| AePAO- | ttcctgcagcccgggggatccTCTTTAT | SEQ ID | tgtttttggcgtcttccatggCTGAT | SEQ ID |
| pGreenII | TTTCTGTTGAATTAACTCCT | NO. 77 | TTATTTACTTCTTTTGGA | NO. 78 |
| 62-SK | G | | ACTCC | |
| AeNYC1- | tcctgcagcccgggggatccAATATTA | SEQ ID | tgtttttggcgtcttccatggAGATT | SEQ ID |
| pGreenII | GCACCTTTTCCCTGTTAATG | NO. 79 | GAAACTAAAAGGGGAA | NO. 80 |
| 62-SK | | | AAATC | |

The dual luciferase assay demonstrated (FIG. 6) that the AeABR1 protein can only directly bind to the promoter of the AeCLH1 gene, but not to the promoters of the AePAO or AeNYC1 genes. Furthermore, upon binding to the AeCLH1 gene promoter, the AeABR1 protein induces the expression of the AeCLH1 gene. In summary, the AeABR1 transcription factor regulates chlorophyll degradation by activating AeCLH1 gene expression, thereby influencing the postharvest quality of okra.

The foregoing has illustrated and described the fundamental principles, main features, and advantages of the present invention. To those skilled in the art, it is evident that the invention is not limited to the details of the exemplary embodiments described above. The invention may be embodied in other specific forms without departing from the spirit or essential character thereof. Therefore, the embodiments should be regarded as exemplary and non-limiting in every respect. The scope of the present invention is defined by the appended claims rather than the foregoing description. Consequently, all variations falling within the meaning and scope of the equivalent elements of the claims are intended to be encompassed within the present invention, and no feature of the claims should be construed as limiting the rights involved.

Sequence list

SEQ ID NO. 1
CDS sequence of AeABR1 gene
ATGTGCTTACTCAACAAGGTGGCGCAGCAGAGAGGTTCAAGTGACTACGCCAGGTTTCCT
ACTGATGGAGATGGTGGCGGTGCCGAAGATGAACAACAATATTCCCAGTTGTCTCAAATCCA
ACAACTCTACCGTCCGATGATGCAACATCGGAACACAGCTGAAATGTTTTTTGGACAACAGG
GTCAATCATCAAACGAGATGTCTGCAATGGTACCTGTTGGATCGGGACAAAGGGCAGCTGCT
GATTGGGGTTATGTTGCTAACATAGCAGGCAAGGTCTCATCTGGTTTTGCACACAGCAGCTC
ACCAGCTCGTTCTTCACCTTCTGGTTCTGGTTCGAGTTCTGGTATATGGGTTGGTAAAAAACG
AGGGCGTGAAGATGAGGCTAGTGTTCATTTCATTGAGTCTTCACCGAGAGGGGTTCAAAGAA
CTTTTGTCGATTTTAGAGGCGAGTCAGCATCAGCTGCAGCTGCCGTTGCTGAAGAAACAACG
AGCATTGTTGCATCGGCAGCAGAAACAACAGTAGGAGCAGCGGCCGTGGCATCCTATGAAG
AAAGCGGCGAGCGAAGAAGAAGATATAGAGGCGTAAGGCAAAGGCCATGGGGGAAATGGG
CTGCAGAAATACGCGATCCTCATAAAGCAGCAAGAGTCTGGCTCGGCACCTTTGACACCGCA
GAAGCTGCCGCAAGAGCCTACGATGAAGCCGCCTTGAGATTCAGGGGAAACAGGGCCAAAT
TAAATTTCCCCGAGCTTGTCAGGTCTGTTCCTCAACCAATGCAGAACTTCCCCGCTACCCAAA
CACCAGTTTCTACTTCTCTTCCAACCCATTTCCAATCTTCTAATCCAACACCATTGCCGTCGTT
TTACCAACCCCAAACTCTTCAGAGCTCCACTGCTGATATGCTGAGGGATTACTGGCAGTACT
CTCAGTTGCTGCAGAGTTCTTCCGGTGTCCATGGACAACAAGATACGAGGTTGATGGAACAG
ATGATCCAATCCTCTCAAATACCTAATATTCAACAGCCATTGTTATCTTCTTCTTTATCACCTT
TCCCTTCTTCATTTGTAGCTTCCTCCGGTTCTTCATCTTCTTCCTCTGCTTATTTTCCTGTGCTT
TTCTCCGAGCATCAACAAATGGACATTTTCGGGCAGCCGTCCAATCAAACTCAGGCAAGTGG
GTCAGATTTTCCAGTGCCACCATGGTCTCATCCCGATCATGATCCTTCATCCACAGGTTCATA
A
SEQ ID NO. 2
amino acid residue sequence of AeABR1
MCLLNKVAQQRGSSDYARFPTDGDGGGAEDEQQYSQLSQIQQLYRPMMQHRNTAEMFFGQ
QGQSSNEMSAMVPVGSGQRAAADWGYVANIAGKVSSGFAHSSSPARSSPSGSGSSSGIWVGKK
RGREDEASVHFIESSPRGVQRTFVDFRGESASAAAAVAEETTSIVASAAETTVGAAAVASYEESG
ERRRRYRGVRQRPWGKWAAEIRDPHKAARVWLGTFDTAEAAARAYDEAALRFRGNRAKLNFP
ELVRSVPQPMQNFPATQTPVSTSLPTHFQSSNPTPLPSFYQPQTLQSSTADMLRDYWQYSQLLQS
SSGVHGQQDTRLMEQMIQSSQIPNIQQPLLSSSLSPFPSSFVASSGSSSSSSAYFPVLFSEHQQMDIF
GQPSNQTQASGSDFPVPPWSHPDHDPSSTGS*
SEQ ID NO. 3
AeCLH1 gene
ATGTCCGCTGCTTTCACAACTTCGGCTACAAATGTTTTCGGGTTCGGAAGCTACACCACCG
TGCTTCAAAAGGTGGATTCGGTGTCGACTTCTTCAGTGCCCGTCCCACCACCAAAGCCACTC
CTTATTGCCACACCTAGTGAAGCAGGAGAATTCCCACTACTCATATTCCTCCATGGTTACCTT
CTTTACAACTCTTTCTACTCTCAGCTTCTCCAGCATGTTGCTTCCCATGGTTTCATTGTTATTG
CTCCTCAGTTATACACCGTGGCCGGACCGGATGCGACCGATGAGATTAAGTCCACAGCTGCA
ATAACCATTTGGTTATCCAAAGGACTACTCCAAGGGTTGCTTCCACAAGATGTTCAGCCAAA
CTTAAGCAAGCTCGCACTTGCCGGTCACAGCAGAGGAGGCAAAGTTGCTTTCGCGCTAGCTC
TGCAGAAAGCCATGACCACGTTGAAGTTTTCGGCCTTAATAGGTGTAGACCCCGTCGATGGA
ATGGATAAAGGGAAACAAACGCCGCCTCCGGTACTCACTTATGTTCCTCATTCGTTCGATCT
TGATATGGCGGTAATGGTTATCGGTTCGGGTCTCGGTGATGTTAGGAGAAATCCATTGTTCC
CTCCTTGTGCACCCAAGGGAGTTAACCATGAAGATTTCTTCAAAGAATGTAGAAAACCGGCA
TGTCATTTGGTTGCCAAGGACTATGGTCATCTCGATATGTTGGATGATGAGACTAAAGGTTT
AAGAGGACGTTCATCGTATTGTTTGTGCAAAAATGGCAAGGCTCGAGAGCCGATGAGAAGG
TTTGTCGGAGGAGCCATTGTTGCTTTCATGAAAGCCTATCTTAATGGAGACAAGACTGAATT
GATTGCTATAAAACATGGGCATGAAACTGCACCGGTGGAGCTTGAAACGACGGAATTTCTTG
TTTAA
SEQ ID NO. 4
AeCLH2
ATGTCTGGTGCTGTCACAGATTCGGGCACCAATGTTTTTGGGTTTGGAAGCTACACCACTG
TGCTTCAAAAGGTTGACTCAGTACCCACTCGTTCAGTGGCTGACCCACCCCCAAAACCACTC
CTAATTGCAACACCTTCTGAAGCTGGAGAATTCCCGCTACTCATCTTCCTCCATGGCTATCTT
CTGTACAATTCTTTCTACTCTCAGCTTCTCCACCATGTTGCTTCCCATGGCTTCATTCTTATTG
CTCCTCAGCTTTACACCGTGGCTGGACCAGATGCAACCGATGAGGTTAAGTCCACGGCTGCA
ATAACCAATTGGTTGTCCAAAGGAGTACTCCAAGGGTTGCTTCCATCGGATGTTCGCCCGAA
CTTAAGCAAGCTTGCACTTTCCGGACACAGTAGAGGAGGCAAAGTCGCTTTCGCACTAGCTC
TGCAGAAAGCAATGACTACGTTGAAGTTTTCGGCCTTAATAGGCGTAGACCCCGTCGATGGA
ATGGATAAAGGGAAACAAACGCCGCCTCCGGTACTTACCTATGTTCCGCATTCGTTCGATCT
TGATATGGCGGTGATGGTTATCGGTTCGGGTCTTGGTGAAGTTAGAAAGAATCCTTTGTTTCC
TCCTTGTGCTCCCAAGGGAGTTAACCATGAAGATTTCTTCAAAGAATGTAGGAAACCGGCCT
GTCATTTCGTTGCCAAGGACTATGGTCATCTCGATATGTTGGACGATGAGACTAAAGGATTA
CGAGGACGTTCGTCATATTGTTTGTGCAAAAATGGGAAGGCTAGGGAGCCAATGAGAAGGT
TTGTTGGAGGAGTTCTTGTTGCTTTCATGAAAGCTTATCTTAATGGAGATAACACCGACTTGA
TTGCTATAAAACATGGGCATGAAACGGCACCGGTGGAGCTTAAAACGGTCGATTTCCTTGTT
TAA
SEQ ID NO. 5
AeNOL gene
ATGGCCACTGCTTCAACTTGCCAATTCTTCACTGATTTTTGGTCTTCAAGGGCTCATCCAA
TCATCTTCTCACCCATCTCCAAACCCTCCCATTTTCAGTCTTACAGCCTTACAGCTAATGTTTC
ACTTCAAAAGAACCACAGCCTCTCCAATCCTCCACTGCTAGTAAAAGCTCAAGCTTCCATCA
ATGGAAAAACGCAACCCATGGTTCCTCCTTACAACGTTCTCATAACTGGCTCAAGCAAAGGA
ATAGGGTACGCCTTGGCGAAAAACTTTCTGGTAGCTGATGACAATGTCATCATTTGTTCAAG
ATCTGCTGAAGGGGTGGAATCCGCTTTTAAGAGCCTCAGAGAAGAATATGGGGAACGTGTG
TGGGGTACTCAGTGTGATGTTAGAGTGGCAGAGGATGTGAAGAATTTAGTTTTATTCGCTCA
AAAGAACCTGGGATACATTGACATATGGATTAATAATGCAGGATCCAATGCATATAGTTATA
AACCACTGGCAGAAGCATCAGATGAAGATCTTATTGAAGTTGTTTCTACAAACACCCTTGGG
TTGATGATTTGTTGTCGTGAGGCCATCAAGATGATGTTGCAACAGCCTCGAGGTGGTCATAT
TTTCAACATTGATGGAGCTGGTTCAGATGGAAGACCAACCCCTAGGTTTGCTGCATATGGTG
CAACCAAACGTAGCGTGGTTCATTTGACAAAATCGCTGCAGGCTGAACTGAAGATGCAAGA
TGTTAAAAATGTTGTAATACATAATCTATCGCCAGGAATGGTGACAACCGATCTTCTAATGT
CTGGTGCAACCACAAAGCAGGTCTACTATGCTTCATATGCTTCTTTTGTTAGTTCAGTTTATA
AAAGAAATTTTCTAATATTTTGTCATTTTCAGGCCAAGTTTTTCATCAATGTTTTAGCAGAAC
CAGCTGAAGTGGTTGCAGACTACCTTGTGCCGAACATCAGGTCGATCCCAGCAAGTGGATCG
TGGAAGCCAACTTATATCCGGTTCCTCACTGGCTTGAAAGCCTACTCCCAGATATTTTCAAG
ACTAGCATTTGGTGCAAGGCGAAACCGATACATGCTTGAAGATTGA
SEQ ID NO. 6
AeSGR gene
ATGGTAACTTTAACTTTTGCTCCTGTTTTGCCTGCTAACACAAGACTGTCGGTGTTTGAAC
TCAATGGACCCCTCTTCGTTTCCAGAAGAAAACCCAAGAAGATGAACCAAGCTGTTGTCCCT
GTGGCAAGGTTGTTTGGGCCAGCTATATTCGAGGCATCGAAGCTAAAGGTATTGTTCTTGGG
AGTTGATGAAAAGAAGCACCCAGGGAAGCTACCAAGGGCTTACACCCTTACGCATAGTGAT
ATAACAGCTAAACTCACTTTAGCCATCTCTCAAACCATAAACAACTCTCAGTTGCAGGGATG
GGCAAACAGAATATACAGAGACGAAGTTGTCGCGGAATGGAAGAAAGTGAAGGGAAAGAT
GTCTTTGCACGTCCACTGTCACATAAGCGGCGGCCATTTCCTGCTAGACTTGTGTGCTAGACT
TCGTTACTTCATCTTCTGCAAAGAACTTCCCGTGGTACTGAAGGCGTTTGTCCATGGAGATGG
GAACCTGCTGAAAAACTACCAGAACTGCAGGAGTCGTTGGTTTGGGTTTATTTTCACTCGAA
CGTGCCGGAGTTCAACAGGGTGGAATGCTGGGGTCCGCTGGTGGAAGCGCAGCACCTCTAG
CGGCGGCGATGGCGGTTGTGATGGGAAAAGACAAGAGGTGTTGGAAGCGAGTGGGAATGG
CCTCAGCGGTGCGGAGATGAGTGTAATTGTTGCTTTCCGCCAATGA
SEQ ID NO. 7
AeNYC1 gene
ATGGCTGCACTGGCAAAACTTCACTTACGTAGTTTCCAGAAAGACCAACCGTTTTCTTTCA
AGGAACAACCGCGGAGCTGGATTAAACGGTGCCGTTTTCTACGGTTGCCCATCATAACGAGA
AACACTTATGGGGTGTACGTTCAAAGGTGTCGGTCATTCAGGGGAGACTATGGAGGGGGAG
CAGTGGGAGAGAAAGAGATGGAAAGTGCGGGAAAAGTTGGGATTTTGATGGAAAATGAAG
TGAAGTTGGAAACAGGAGATGGGTTTTGGAAATCTTTGAAATCTGCTGTTTTGGGGGTTAGT
AAATTCGATTCCCAATCTCAAGATGAATATGAAAAAACTGTAGCTAAAGTTGAAGGAGTTTT
CTCTTTGATATCTATGCAAATAGGAAGATATGTAGTGACCATGATGAGCACTGGAGTGATAC
TCTTGACTGGTTTTCAGCTGTCAGGTGGAGACAGTGAGATGAATGCATTGATTTGGTATAGC
TGGCTTGGAGGGATTATAATTGGAACCATGATTGGTGCTAATATGGTTCTAGAGGAACACTG
TCGAGCCGGTCCGAGAAATGTTGTTATAACAGGAAGCACAAGGGGATTGGGAAAAGCACTT
GCTCGAGAGTTTCTTCTTTCGGGAGATTGCGTGGTTGTTACTTCTCGCAGTCCCGAGTCTGTT
CATGTGACTGTCAAAGAACTTGAGGAAAACCTCAAGGAAGGTATGACTGCAGGTGGTTCAT
TTAGTAAGAATCTGGGACGAGCAAAAGTGGTCGGCATAGCATGTGATGTTTGTGATCCTAGT
GATGTTGAAAAATTAGCCAACTTCGCTGTGAATGAACTCGGGTCTGTTAACATTTGGGTAAA
TAATGCTGGCACTAACAAAGGTTTTAGACCGCTGCTACAGTTCAGCGATGATGATATTCGAC
AGATCGTCTCAACAAATCTTATCGGATCTATTCTCTGCACTCGGGAAGCTATGCGCATAATG
AATAACCAGCCAAAGGTGGGACACATTTTTAACATGGACGGAGCAGGTTCCGGGGGATCTA
GCACTCCTTTGACTGCTGTATACGGTTCGACTAAGTGTGGTCTTAGGCAGCTTCATGTATCAC
TTCTGAAGGAGTGCAGACGTTCTAAAGTCGGAATCCATACTGCATCTCCCGGCATGGTCCTA
ACTGATCTGCTCTTAAGTGGTTCGACCTTAAAGAACAAGCAAATGTTTAATGTCATCTGTGA
GCTTCCTGAAACTGTTGCTAGAAGTCTCGTTCCACGATTGCGGGTTGTAAAGGGGACTGGGA
AGGTCATCAACTACTTGACCCCACCTCGAATATTACTAGCTTTAGTCACCGCCTGGCTCCGAC
AAGGTCGCTGGTTTGATGAACAGGGTAGGGCCTTATACGCAGCAGAGGCGGACCGCATTCG
AAACTGGGCTGAAAACCGTACACGCCTCTCGTTCACAGATGCAATGGATATGTACACAGAA
AACACCTGGGTGTCTGTGTTCTCACTGTCGGTTGTTTGTGCCTTCATTATCCTTTCTAGCACG
AACAGCGCCTTTCCCGGCACCTGA
SEQ ID NO. 8
AeHCAR
ATGACTTCAGCTGTTTCCTGCTTCTTAAGACCCATCTTATCTTCTTCTTCAAAAGAATCAA
ACCCCGATGTGAAGAAAACAGTGAAGCTGAGAGATGACTGGAGGAAACGCTCTAAGCCTAT
ACCCCCTGGAGGGACATATCCAGCCAAAGACCATTGCAGTCGATGTGGGTTGTGTGATACAT
ATTATATTTCCCATGTTAAGGATGCTTGCGCTTTCTTAGGAGATGGAATGTCCAAAATTGAA
AGCTTAGAACCTGTAGTTCACGGTAGAGGGAGGAAATCAGATTCTTTGGATGAGACATATTT
AGGAGTCTATGAAAAGCTGCTATATGCTCGTAAAGTTAAGCCTGTAGAAGGAGCTCAGTGG
ACTGGGATCGTTACAATCATTGCAATAGAAATGCTGAAATCAGGCATGGTTGAGGCTGTTAT
TTGTGTTCAGAGTGATCCAGATGACAGACTGTCCCCAAGGCCCATTTTAGCTAGGACACCAG
ATGAAGTTTTAGCTGCTAAAGGAGTCAAACCAACATTGTCCCCTAATCTGAATACCCTCGCA
CTGGTTGAGGCAGCAGGGGTAAAGCGTCTCCTTTTCTGTGGTGTTGGTTGCCAAGTGCAGGC
GTTAAGATCTGTGGAGCACCATCTAAATTTGGAAAAGCTTTATGTGTTGGGCACCAATTGTG
TTGATAATGGAACTCGAGAAGGTCTAGATAAGTTTCTAAAGGCTGCGAGTAGTGAACCTGA
AACTGTTCTGCATTACGAGTTTATGCAAGATTACAAGGTCCACTTAAAGCATTTGGATGGTC
ATATTGAAGAGGTTCCTTATTTCTGTCTACCAGCAAACGACTTAGTAGATGTTATTGCACCTT
CATGCTATAGTTGTTTCGATTACACAAATGGATTAGCGGATCTGGTGGTTGGATATATGGGC
GTACCAAAATATTCTGGAATCAGCATGACGCAGCATCCGCAATATGTTACCGTTAGAAACGA
ACGTGGCAGAGAAATGCTGAGTTTGGTTGAGAACCTTTTGGAGATCACTCCAACAATTAGTA
GTGGTAATCGGAAACCATTTGTGATGGAGACTGTTAAAGCAGATGACGATGCTAAGTTCGGT
ATGTATGGCATCTTCTGTTTAGCCATGGAAACAATTTTTGTTTCAACCGCAGTGCATAAACAT
TAAGCTTAAATGCATTATATGGAGTCCTTGTACAAGTGATGATAACTGCATATAGCTGAAAC
TTACTTAGAGGATTCATGGAGTATGTACTACATTATACTGTGTATTTTGCCTTCAGGAAAGGG
TCCTTCTCAACCTGCCCCAAGGTTGATTGGGAATTTGATAGCTTTTGTGCTCAACTTGATTGG
TCCAAAGGGTCTGGAATTTGCTCGGTATTCACTTGATTACCATACTATAAGAAACTACTTGTA
TGTGAACCGTGCCTGGGGGAAACAAAGAGCTGATAGGCATATTCCTTCATACGCAAAGAAA
CTTGTGGATGCTTATAACCAGAAGGGTGATATTGACCAAATGCTTACCCGTCAGTAA
SEQ ID NO. 9
AeRCCR gene
ATGGCTGTCGTCTTCCCACGTTTTCTAAATTCCCCTCTCCCTCACTCTTCACTTGCTTCTTC
TTTGGCTCCGCCTTCACCCAGGCCTCAATTCTTTGCATCTTCTGCACCATCATCGTCATCTCCC
ATGGAAATCGCCCCCCAAAAGTACACGGATTTCACATTCGTCTCTGCCCCACATAGGAACCT
TATGGTTGATATGGTGTCGACGATGGAGAATCGTCTGGACTCTCAGCTCAAACCATGCACTC
TACCCTCTGATATACAATATTACTACACTCCAAGTGGGAACTCCCATGGCTCACTTTATATCA
GATCAGGCCATACCTCTTCTCCGGTTGATTTCGTTCTAGGAAGTTGGATAAACTGTAAGATG
CCAACAGAGGGAGCACTGAACATAACAAGCCTTTCAGCATATCTGAATGCTTCCACAGATGC
CCCGAATTTCCTGATTGAACTCATACAGAGTAGCCCTACATCACTTGTTTTCCTTCTGGATCT
GCCCCCTCGAAAGGACCTGGTCTTATATCCCGAGTACCTTAATACTTTTTACGAGAACACCC
GACTTGAATCACTTAGGCAAACGTTTGAGAAACTCCCAGAAGTTCAACCTTACTACTCCTCT
GCTCTTTACATTCGATGCCTTACATCCCCAACTTCAATCATGATCCAAATCAATACAGAGGG
ATCCAGAGAAGGACGCATGGAGGAGATTATAAAGGATCATGTACACCCGGTAGCAAAAGAA
GCATTGGGGATATGGTTGGACCGATGCGCTTGTGGACATAGAAATGTGGACGAGGAAGGAA
AGGCATATTTGGAGAAGAGGGATGGGTTGATTAAGAACAAGACAATTGAGATTGATATAGG
TTCAAGCTTCCCAAGATTGTTTGGGCCCGACATAGCAAATCGTGTATTAGAGGTGATAAGGG
AAGCATACAATGGCTGA
SEQ ID NO. 10
AePAO2 gene
ATGGCAGCCTCTCTTCTTGCTACCATCCCTACAGCCAATTTGGCTACTCCTTTTATAACAT
CTTCGGAAAGCAAAACCCATCTCTTTTTATGTCCACGGAAGAACCTTGCTTCAAGAAGATTC
ACGAAGCTTACACCTTTACACGTGGCAGCCTCTCCATCATCATCATCAGCAGCAGAACGAGC
TTCAGATCAGAAACAAGATGAATTCAGGGTAGGGGATGAATTTGGTGAGGAGAGTTCAAAG
TCTAAGTTCTCATGGAAAGATCATTGGTACCCGGTTTCTTTGATTGAAGATTTGGACCCATCT
TTACCCACACCATTTCAGCTTCTGGGTCGTGACTTGGTGCTTTGGTTTGATAAAGGAACGAGC
CAATGGGTTGCTTTTGATGATAAATGCCCCCATAGGCTTGCCCCTTTGTCCGAAGGGAGGAT
AGATGAGAATGGGGACTTGCAGTGTTCATATCATGGATGGTCTTTTGATGGATGTGGATCTT
GCACTAGGATTCCTCAGGCTGCGTCTCAAGGTCCCGAAGCTCGTGCGAATCGGTCTCCAAGA
GCATGTGCTACTAGGTTTCCTACAATGGTTTCTCAAGGCTTGCTTTTTGTTTGGCCTGATGAG
AATGGGCAGGAAAGAGCAAGTGCTACCAATCCCCCCATGTTGCCTGATGACTTTAGCAGACC
CGAATTTGCGACTGTGAATATCCAACGTGATCTGTTTTATGGTTATGATACCCTCATGGAGA
ACGTCTCCGATCCTTCCCACATCGATTTTGCTCATCACAAGGTTACTGGGAGGAGAGACAGA
GCAAAGCCTTTGCCGTTTAAGGTGGAATCTAGCGGCCCCTGGGGATTTGGTGGATCAAATGA
GGATAACCCAAAGATCAGTAGTAAGTTTGTTGCACCGTGCTATTATATGAATAAGATAGAGA
TAGACACGAAGCTTCCAATAGTTGGTGAACAGAAATGGAAGATATGGATATGTTCATTCAAT
GTACCAATGGCTCCAGGGAAGACACGATCAATCGTTTGCAGTGCCCGAAACTTCTTCCAGTT
TTCTGTGCCTGGGCCTGCCTGGTGGCAGCTGGTTCCGAGATGGTTTGAACACTGGACATCAA
ACGAAGTTTATGATGGAGACATGATTGTACTCCAAGGTCAAGAAAAGATATTTCTTTCAAAA
TCAATGGAGGGTTCCAAGGACATCAATCAGGAGTATTCGAAGCTCACATTTACACCGACACA
AGCTGATCGATTCGTCTTGGAATTCCGGAAATGGCTAAGGCGTCACGGAAACAGTCAACCCG
AATGGCTCAACACCACCATCCAACCTCTCCCGTCAACAGTATTATCAAAACGCCAGATGCTT
GATAGATTTGAGCAGCACACACTCAAGTGTTCATCATGCAAACAAGCTTACACGTCGTTCCA
AACATGGAAAAAGATCTTAGTAGGTGCAACTGTTGCATTCTCCGCATCAGCCGGCGTCCCAT
CAGATTTGCAGTTTCGGGTTGTTTTAGCGGTGCTTGCAGTGATTTCTGCAGGTTTGGCATTTG
CATTACATCAACTCGAAAAGAACTTCGTTTTTGTGGATTACGTACATGCTAAAATAGATTAG
SEQ ID NO. 11
AePAO4 gene
ATGGCAGGGTTTCTTCTTGCTACCATTCCTACGGTCAATTTGGCTACTCCTATTATAACAT
CTTCGGAAAGCAAAACCCATATTTTTTTATGTCCACGGAAGAGCCTTGCTTCAAAAAGATTC
ACGAAGCTTACACCTTTACACGTGGCAGCCTCTCCATCATCAGCAGCTGCAGAACAAACTTC
AGATCAGAAACAAGAAGAATTTAGGGTAAGAGATGAATTTGGTGAGGAGAGTTCAGAGTCT
AAGTTCTCATGGAAAGATCATTGGTACCCGGTTTCTTTGGTTGAAGATTTGGACCCATCTTTA
CCCACGCCATTTCAGCTTTTGGGTCGTGACTTGGTGCTTTGGTTTGACAAAGGAAGGAACCA
ATGGGTTGCTTTTGATGATAAATGTCCCCATAGGCTTGCTCCTTTGTCCGAAGGGCGGATAG
ATGAGAATGGGCACTTGCAGTGTTCATATCATGGATGGTCTTTTGATGGATGTGGATCTTGC
ACTAGGATTCCTCAGGCTGCGCCTCAAGGTCCCGAAGCTCGTGCAAATCGGTCCCCGAGAGC
ATGTGCTACTAGGTTTCCTACAATGGTTTCTCAAGGCTTGCTTTTTGTTTGGCCCGATGAGAA
TGGGCAGGAAAGAGCAAGTGCTACCAAGCCCCCCATGTTGCCTGATGACTTTAGCAAACCA
GAATTTGCAACTGTCAATATCCAACGTGATCTGTTTTATGGTTATGATACCCTCATGGAGAAC
GTCTCGGATCCTTCCCACATTGATTTTGCTCATCATAAGGTTACTGGGAGGAGAGACAGGGC
AAAGCCTTTGCCGTTTAAGGTGGAATCTAGTGGCCCTTGGGGATTTGGTGGATCAAATGACG
GTAACCCAAAGATCAGTAGTAAGTTTGTTGCACCGTGCTATTATACGAATACGATAGAGATA
GACACCAAGCTTCCAATAGTTGGTGAGCAGAAATGGAAGATATGGATATGTTCATTCAATGT
GCCAATGGCTCCAGGGAAGACACGGTCAATTGTTTGCAGTGCCCGAAACTTCTTCCAGTTTT
CCGTGCCTGGGCCTGCCTGGTGGCAGCTGGTTCCGAGATGGTTTGAACACTGGACATCAAAC
AAAGTTTATGATGGAGACATGATTGTACTCCAAGGTCAAGAAAAGATCTTTCTTTCAAAATC
AATGGAGAGTTCCAAGGACATCAATCAGGAGTATTCGAAGTTCACATTTACACCCACACAA
GCTGATCGCCTCGTCTTGGAATTTCGGAAATGGCTAAGGCATCACGGCAACAATCAACCTGA
ATGGCTCAATACCACCATCCAACCTCTCCCGTCAACAGTATTATCAAAACGCCAGATGCTTG
ATAGATTCGAGCAGCACACACTCAAGTGTTCATCATGTAAACAAGCTTACACGTCATTCCAA
ACATGGAAAAAGATCTTAGTAGGTGCAACTGTTGCATTCTCCGCATCAGCCGGCATCCCATC
AGATTTCCAGCTCCGGGTTGTTTTAGCAGTGCTTGCAATGGTTTCTGCAGGTTTGGCATTTGC
ATTACATCAACTCGAAAAGAACTTCGTTTTTGTGGATTACGTACATGCTGAAATAGATTAG
SEQ ID NO.12
AeABR1-qPCR-F
CCGAGCTTGTCAGGTCTGTT
SEQ ID NO.13
AeABR1-qPCR-R
AAACGACGGCAATGGTGTTG
SEQ ID NO.14
AeERF114-qPCR-F
CGGCGGAGATAAGAGATCCG
SEQ ID NO.15
AeERF114-qPCR-R
ACCTAAGAGCCGCTTTGTCG
SEQ ID NO.16
AeERF105-qPCR-F
TGGGTTTAGCTCTGGTTCCG
SEQ ID NO.17
AeERF105-qPCR-R
ATCCGAGCGCCTTTCTTGTT
SEQ ID NO.18
AeActin-qPCR-F
TCGGTGAGAAGCACAGGGTG
SEQ ID NO.19
AeActin-qPCR-R
GGTTGGGATGGGTCAGAAGG
AeABR1-pCV-eGFP-N1-F
SEQ ID NO.20
cgacgacaagaccgtcATGTGCTTACTCAACAAGGTGGC
AeABR1-pCV-eGFP-N1-R
SEQ ID NO.21
gaggagaagagccgtcgTGAACCTGTGGATGAAGGATC
SEQ ID NO.50
AtActin-qPCR-F
GTCGTTGCACCACCTGAAAG
SEQ ID NO.51
AtActin-qPCR-R
TGAACGATTCCTGGACCTGC
SEQ ID NO. 70
promoter of AeCLH1
TTTTTGTCTGAACTCCGTTAAGGACATATCTCATGACAACCTCTCTGGCTCTAGTGTCAGC
TCTATCATTTCCCACCAGTCAATGGTGTCACCCTTGAATAAGGCAGTGACGCACTCGAGCTT
ACCCTATGGGGTAAGGTATAACTGTCTGAGGGTGTGGAGAGTCTCCCTAAACCATTAGTCTA
CTGTTATGAGATCGTCATCCTCTGAACTGAAGAATTTGATGGTCTTATATCTACGGAGTCTCT
CTACTAGAAACTCTTGGGATACTACAACTGGCTAGGCTGGAGGTGTTGGTGGGGGAGGTGGT
GTAGTTGGGGCGCTAGGTATGGTCCTAATGATCTACAAAGAGTTTAGGTGAAAAAATTATAA
AAAATATGAAGAAATATATAATTTATTTATAAATTTTATAAATTTTCCTAAAATTATGAAAAT
AATAAAAATAAAACAGAAAATATTTTAAGAATTCTAAATGATATAAAAACAAAAAATGTAC
AAAAGTATAAAATTCATAAAAAAATTAAAAAAGTAATTACCAACTATAAGTTTCTTTTATAA
AAAAATTACAAAAAAGTAAATTTATATTGTAGATGAGCACGTATCGACCTTGCAAGTTGAAT
ATTTCTTAATTACATGGTTTCAACGAGTTTCATGGGGAAATGCTCACGTGATGCAGTCTTAAT
ATACTGCTAAAAAAACGTTATCAGTTTTATTATTGTTTAAATAACAAATTCAGTAAAAAATT
ACCGGAAACATTATTAAGCGCTAACGGTTTTGTATTATTGAGCTTTTTACCGAATTTGGTAAA
AATGACCGAAAATTTTTAGATGCTACGGTTTTGTTATTGTTGGACACTTTTACCGGAAATGTT
AAAAAATTGATGTTTTAGATAAAATAATAATATTTTAGGTAATTTTTTATCAATTTTATTATT
TAAACAATAACCAAACCGGTAACGTCTTATTTTAGTCTTATATTAAAGTGGCACACTGTAAA
ACTTTCGAGTTTCATGACATTGGCAAGTTTCGGTGCATGAAGACGAAACAATTATTTTTGTA
AAAACAGATTTTTTTTACCTTTGAGCTTATTTTTATAAAAGAAATTAATTTTAATTATTTTGTT
TATAATTTTATGATTTTATAATTTTGTTTTCATATATATATATATATATATAAATATAAAATAT
ATATATATTTTCAATTTTAGTAATTTTATAAAAATATTTTTAATAAAACTTTAAATATATTTTT
ATAATTTTTCTTATTTTCATAATTATTTACAATAGACTTAATAAAAATGTCATATAGTCAAGA
ATTATTATTCATGTATTATACCTTAAAAATATCCAACTTAAGAAATATCGAAAAAATCACAC
TCACATCTGACTTGAGTAGAGTTTAGGAGATCTCATCCAATCCGAAATTCGATCAAATTTTTT
GGTCAAAAATCACACTCAACCTATATATATACAGGGTTAGAATCTAATTACATAGTGTAAAA
CTAAAAATTATATAGTTTTAGTTTTACACCATTTTAGGATCGTTAGATGAGGGGAAATTAAA
GACTGAGATTAACAATAGGAAATGCGAAGACACTCACTAGAAAAAAAATAAGAAGTTAATC
TCAACATTGATTTATCTCTTCCAACGATATTAAAAGTGTAAAACTAAAAAAATTACTATAAA
TGTAAGTGAATAATAACTCATATATATATATATAGATAGATAAATATTAAATAAAATAAGAA
TTTAATTAAAAAATCTTATTAAAATTATCTCTAAATTTATAAAAAATAATTATAATATTACAC
CAGGCTTGATCTGATTATTTATCACGATCCGACCCAATAATACCTCTAAAAAACCCAATCTG
AATCCAATACGACTTAAGAAATATCCGAACTGGCAATTCACCTACCTTATCTCGTTGTTTCCC
ACCTATAAACAAACAACAGGAACTGAAAAACCAGAAGCAGAAGAAGCAACAACAGATTGG
AAGAC
SEQ ID NO. 71
promoter of AePAO
CCTCATTTTCTGTTTTACAGGTAGTTTGAGCTACTAATTACTAGTCTAAAATCCATTTCGAT
TTGTTCTATAAAATGTATGGATTAACATGTAGAATTTCGTATTGATATCGGAAATGTCAACA
ATTTCTTTCACATGTTATAGGTCTCTTGTCTTTCCTTGTTGAAGTTGGTTTAGAATCAGAATGT
CACGAGCACGAAATGTCTTGGTTGCTTCTGGCTTACTAGCTTTTGTCGCTGCAGGCTTAGCAT
TTCCGTTTTATATGGCGTAAGTATAATTCATTTTTCAACAACAATGTGTTGTTGGGAGCTATA
TGGTTATCAACTTTCAAAAGTTCAATAACGGTTTGTATCGTCCAGGACATCTAGGAAACCTG
TGGATTCATCTAAACCACTTCCTCCGCAGGCAACTTTTCGAGGGCCTTACATAAATACTGGTT
CACGTGATGTTGGACCTGATCACCAAATTTACCCGAAGAAATGATCTTTATTTTCTGTTGAAT
TAACTCCTGTTAACTGTAAGTTAACCGTATGCTCCTTTCGTTCGTGCACCACGTGAATTGACT
ATGCATTTGAAATTAGGCTTTCTTTCCAACTATTGGGTGCGTATGTGGAGGCTGAACTAGGA
ATTCGCTTTGCTTCCTGCAGCATGATGAGTATTGTGTGACAACTGAACATGAAAAATTTATTT
TGTAAGAATTTAGATATGAAAAATTGTTATGCTTTCTCTGTGGAAATCAAGTGCATAACATG
AAGAGGAAAAGAGCAGAGGCTATACTTTGTTTTGCTTTTATTTTTGTCATGATCTTATGTTAC
CTTTTATTTTCTTTTCCCCCGATTTGTCTCGTGTTCTTATTTCTAACTTAAGATGAAATATAAG
CATCATCTATTAAAATGATGTCGAGTAGTTTACGCTTCCGACAGGCGTGACTGTGTTCGATAT
GTTACGGCCGAGACTTGTTCATGGTAGTAGGTTCTCATCTAATTGATTCGGCCGTAAAAGAT
TTCTTCTAAGTTTCTTGATAAAGCCCAGGATTTGTTAATTTATTGCTTTGTGGTTGGGGCTGC
CTGCAGTTCTTGCAACAGAGCATACCTTTCTTGTATCTTTATACAGGGTGAGGGTGGCTCTGG
CCCTCTTTTTGGTTTAAGAACTTCTTAGATCATTGTTAAATTAGGCAAATTGAGCACTGAATT
TATGCAAATAGGCCCTCATCATCTTTTTTAACACTTTATGTTACAGTATCTGACGTGTGGCCT
TCTTTCTTTATTCCTCCGAATTTCATCAACTCTTTCGTTCTTTCTTTTCCCAATTTTCTTAGTAA
CCAAACAGAAAATGGAAGAAAAATAGATGGATATAATAAAACATAATCCTTTTACCATCTT
CGTAGTCCATTTGGTTTTCGTTTAAGCCATCATCTTGTTCAATATATTCTTCTTCGTTCTTGTT
TTTTAAATTATTTTTATTGAAAAATCATAAATCTCAAATACTTTAGTTTTAAAAATTCGTCTA
TTAAAAAAAATCATAATAATGGATAATAATTTTAATTTTATTTATTTAATTTGAGAAATTCTA
AGTCTAAAAGAAGTTACAAAATCGAAGAATTCATAATAGTGGAAAATGTTTTTTCAACGGGC
ATAGCAAATTAACTTAAAATAAATGTTTAAAAGTAAAAATATATATATTTAAATATTTATTTT
ACTATTAAATTTTTATATAAAAAAAAAAATATGTCAAGTAGATAATTAATCACACGCGCTGA
AATGAAAAGCTTACGTGTGAAATCATTCCAGCTATTATTGCCACGTTGAGTGTGAATACAAG
CATTAGTTTTCCATTGTCCTTTCTAGTTTTTCCTGGTGGCCAAGACGAGTTCAATTCTTGCACT
CAACTCAAGTTTTTTTTTTCTTCTTCATCTTTCATTTTTCTTTCTAATACAAGAACCCAGCTTC
AGTTTCTTAAGGAGTTCCAAAAGAAGTAAATAAATCAGAAAAAGAAAAAAA
SEQ ID NO. 72
promoter of AeNYC1
TTAAAACTTAAACGGGATACCAAAATAAAACAAGAACTAAAGTTTTAATGTCGGTACTAA
GATGAAACAAAAACTAAACTTTAAATATGATCTTGAAAAATTAAAAGTTTAAGTACCAAAA
CAAACCAAAATTAACAGTTTAAGAACTGAACTCGTAATTGACCGAAATAATAATTCCAAAAT
ATAGAGGACAAATCTGACAATATTAGCACCTTTTCCCTGTTAATGCTGGAAACTACCACATC
GGTACATATTTTTGTCACCTGTAAATTTCCATATTTGAAAGTTAAAACCAGCAGTATATTTTT
ACTCAACATTATAGACATTTCCTTGGATTGGATTGATTTTAAATATAAAAATTATATAATTAA
TAAGTCGATGAGGATCGAATATAATATTTTATGTTATATGATACGAACTTGTAACATTTTAA
AAATGTGTTATATCATCAATCTAATATATAAAGTTATATAAATTATATGAGAATTTAAAATC
GTGTAAAATCGAGATCCTAATCGAACATCGTTTCGATCTATCCGAATTCGATAAAGACTCAA
GTGAGATAATAAAAATGAGGAAGAATATCTGGGTGTGATGGAAAGCACATAAAAAATATTA
AAATATATAATAATTTTAAATTTTCAGAGGACGACTGTCTCGTTTATCTATTTGAATCCTTCC
TACAAAACATCCTTCAACTTGCTTCATTAGTCTCAAAGCTCTCAACTGGTATTATTAAACCCC
TTTGATCCTCTATTTATAATCATTAGTTTTCTTAGATAGATAATCATGAATTATTTTCTTAGGA
AGATCCTAAACCAATAAGGTGGTGTTTGGTTCAAGGATTGTGGGATTCCCATGGGGGAGAAT
CTCACATTCCCATGTTTGGTTGGCCGGAAATCTCATTCCCAGTAATGTTTTATTACCAATAGT
AGATTTTTTGGGGTATATTATCAGGAAGTCATTACCTCCATTCCCTTAGGTATTGTAACTTAC
GGCGTTTGGACTAAAATGCCCTTAATTTTTTTTTCAAAAATATCATTCTTCTGTCATTGAATA
ATAAAAATTATACGAAAGTTAAATTACAAATTTATCCTTAAATTATTTTAGATTTACAAAAA
AAATCTATAATTAAAATTTTATATGAAGTTCAGGGGTTAATTTTTTTAACCTTTCATTAAACA
TTTTTTATATTTTTAATTTTGTAAAAAAATATATAAATATTATGAATTTAATTTTTTAAATAAA
ATTCATAAATATTTTACAATTTGTATTTATACAAATATATATAAGGACATTTGGTCAAATATC
ATTCCAATAATCTTTATTCCTTTAACCAAACACGGTAATCTTACATTCCTGCTAAACCAACCA
AACGCAAAATACAACATTCCCAGTAATCCTATTCTCTGGTAATCTTTTATTCCCACTGGTAAT
CTTACATTCCGTGAACCAAACGCACCCTAAAGGGTAAACTAATTATTTTTAGTCTAATTTTTT
TAAAATTATACTTAAAATTATATTTTAATTATCTGTTAAAAAATTTAACGATAATATAATATG
TTTAAAAAAATTATATTAAAAAATAATAAAAATACTATCAATATCACGTTAATTTCTAACAG
TAGATGATTAAAATAAATTGATTTTAACAATGAATGATCGAAACATAAATTAAATAATATCT
GAGTGACCAACCATATATAATAAAAGCCTGAAAATATAAGGCGTAAGAGCGTCACCTCTTTC
AAACCAGCACGACAACGAAAATAGCGACACAAAACGCCACGTGTCGACCTAAACGACAGC
AGATTAGGTTCTGCTCAACGTGTGGTCATCACGCTCGTCTGCCTCGAAAGGCAAATTTTAAC
AGGAGAAAATAATCATCGGAAGGAAGGTTCTTTTTAATTCTTTCCTTGCCGATAAAGTTGGA
GAAGAAGAAGAATAAGTACTGTTTAAAGATTCGATTTTTCCCCTTTTAGTTTCAATCTCCGCC
AAAA
SEQ ID NO. 81
AeERF114 gene
ATGCATTACCGAGGTGTTAGACAAAGGCCATGGGGGAAGTGGGCGGCGGAGATAAGAGA
TCCGATCAAGGCCGCTCGCGTCTGGCTCGGAACCTTCACTACAGCGGAAGCGGCTGCATTGG
CTTACGACAAAGCGGCTCTTAGGTTTAAAGGAAGCAAAGCAAAGCTAAACTTCCCTGAAAG
AGTTCATCGAACATCCGAATCCGGTTGTCTAACAACTAGCCTTGAAGTCGGACCGGAGGCGC
CGCCGACACTTTCACATCCCAATATATCCGATCAGTACGCACGAGCACAGGCAGAGCTTCTA
GCCGGTGGAAGCAATGGTTTAAACTATTGTTTACCAGGTGGCTGTGGTGGAGCAGTCGGAAC
ACCCTTTACTTCACACTCCACAACCCTATCTTCATCTTCATCGACGTTAACATCTTCATCTGCT
TACGGTGGTTTATCTCCGACACCGAGTTCGGATCCTCCGAAGCATAAGAGAGACTAG
SEQ ID NO. 82
AeERF105 gene
ATGGGGTTTTCTGAAGAAACATCTGCTCAGTTGGAATTCATAAGGAGACATCTCTTTAAT
GATTTTGTTTCCATGGAAAACTTTATGGTTTCCAATGTTGATACCACGAATCGGAGAAAGCA
ATCGACGCTCAGCCAACGTCGTCCTTGTATCAACGTTATGATCCCTCCGGCGAACGTTAACG
TGGGTTTAGCTCTGGTTCCGGTTCCGGTTGAAACGTGCGAGGAAAGGCATTACAGGGGAGTG
AGACGACGGGCGTGGGGAAAGTTTGCATCTGAAATCAGAGACCCGAACAAGAAAGGCGCTC
GGATTTGGCTCGGAACCTTCGACACCGCCATTGAAGCTGCAAAAGCGTACGACAGAGCTGC
GTTTAAGCTACGGGGTAGTAAAGCCATATTGAATTTCCCTCTTGAAGCTGGAAAATTGAATT
CCTCGTCATCTGAGGATAATTCCTTTTCCAGGAAAAGGAGAAGAAGTGAAGAGGAGGAAGA
AAAGAGAGTGGAGTTGGAGTGTAAAGTAGTGAAAAGGGAGGAGCCGATGGCGGAGGCCCC
CAAGGAGGCGGTGGCGGAAAGTGTAACGCCGTCGACTTGGACGGGTTTGGGGGATAACGAA
GATATGAACGAAATATTTAGTGGTCCTTTATTATCTCCATTATCGTTACACCCATGGTTTGGG
TATTCTAGGCTTGAGGTTATGTAA

1. A method for reducing chlorophyll degradation in a plant, comprising: decreasing the ability of the AeABR1 gene to produce chlorophyll, thereby reducing the expression level of the AeABR1 protein as to decreases the expression of genes associated with chlorophyll degradation induction.

2. The method according to claim 1, wherein the AeABR1 gene comprises the nucleotide sequence as SEQ ID NO. 1.

3. The method according to claim 2, wherein the AeABR1 protein comprises the amino acid residue sequence as SEQ ID NO. 2.

4. The method according to claim 3, wherein the chlorophyll degradation-related genes are any one of as below: AeCLH1, AeCLH2, AeNOL, AeSGR, AeNYC1, AeHCAR, AeRCCR, AePAO2 and AePAO4 genes.

5. The method according to claim 4, wherein the AeCLH1 gene comprises the nucleotide sequence shown in sequence of SEQ ID NO. 3; the AeCLH2 gene comprises the nucleotide sequence shown in sequence of SEQ ID NO. 4; the AeNOL gene comprises the nucleotide sequence shown in sequence of SEQ ID NO. 5; the AeSGR gene comprises the nucleotide sequence shown in sequence of SEQ ID NO. 6; the AeNYC1 gene comprises the nucleotide sequence shown in sequence of SEQ ID NO. 7; the AeHCAR gene comprises the nucleotide sequence shown in sequence of SEQ ID NO. 8; The AeRCCR gene comprises the nucleotide sequence shown in sequence of SEQ ID NO. 9; The AePAO2 gene comprises the nucleotide sequence shown in sequence of SEQ ID NO. 10; The AePAO4 gene comprises the nucleotide sequence shown in sequence of SEQ ID NO. 11.

6. The method according to claim 1, wherein the chlorophyll degradation-related gene is AeCLH1, wherein the AeCLH1 gene comprises the nucleotide sequence shown in sequence of SEQ ID NO. 3.

7. The method according to claim 6, wherein a siRNA is provided to reduce the coding capacity of the AeABR1 gene, thereby reducing the expression of the AeABR1 protein.

8. The method according to claim 7, wherein the siRNA comprises sequences as shown in SEQ ID NO. 40~SEQ ID NO. 41; SEQ ID NO. 42~SEQ ID NO. 43; SEQ ID NO. 44 to SEQ ID NO. 45; SEQ ID NO. 46 to SEQ ID NO. 47, and SEQ ID NO. 48 to SEQ ID NO. 49.

9. The method according to claim 8, wherein the siRNA comprises the nucleotide sequences shown in SEQ ID NO. 42 to SEQ ID NO. 43.

10. The method according to claim 9, wherein the plant is okra.

11. The method according to claim 10, wherein the plant is a fruit of the okra plant.
